(19) United States
(12) Patent Application Publication
(10) Pub. No.: US 2024/0165597 A1
(43) Pub. Date: May 23, 2024

(54) STERICALLY HINDERED N-ALIPHATIC N-HETEROCYCLIC CARBENE CATALYSTS AND METHODS USING SAME

(71) Applicant: Rutgers, The State University of New Jersey, New Brunswick, NJ (US)

(72) Inventors: Michal Szostak, Newark, NJ (US); Md. Mahbubur Rahman, Newark, NJ (US)

(73) Assignee: Rutgers, The State University of New Jersey, New Brunswick, NJ (US)

(21) Appl. No.: 18/278,932

(22) PCT Filed: Mar. 2, 2022
(86) PCT No.: PCT/US2022/018445

§ 371 Date: Aug. 25, 2023

Related U.S. Application Data
(60) Provisional application No. 63/155,492, filed on Mar. 2, 2021.

(51) Int. Cl. B01J 31/18

(52) U.S. Cl. CPC B01J 31/1815; B01J 31/184; B01J 2231/321; B01J 2531/16; B01J 2531/17; B01J 2531/18; B01J 2531/824

(57) ABSTRACT

The present disclosure relates in part to sterically hindered N-aliphatic N-heterocyclic carbene (NHC) ligands, which are readily synthetically available from inexpensive starting materials. The present disclosure further relates to NHC catalyst complexes comprising transition metals such as Cu, Ag, Au, and Pd. Furthermore, the present disclosure provides methods for using the catalysts described herein in a number of organic transformations, including alkyne hydroboration and hydration, in addition to C—O, C—C, and C—N coupling reactions.

### CROSS-REFERENCE TO RELATED APPLICATIONS

The present application claims priority under 35 U.S.C. § 119(e) to U.S. Provisional Application No. 63/155,492, filed Mar. 2, 2021, which application is hereby incorporated by reference in its entirety herein.

### STATEMENT REGARDING FEDERALLY SPONSORED RESEARCH

This invention was made with government support under GM133326 awarded by the National Institutes of Health and CHE-1650766 awarded by the National Science Foundation. The government has certain rights in this invention.

### BACKGROUND

ItBu (1,3-di-tert-butylimidazol-2-ylidene) is a bulky N-alkyl N-heterocyclic carbene (NHC) that is presently regarded as one of the most useful NHCs in organic synthesis. The importance of ItBu is reflected in its numerous applications in transition-metal catalysis using a plethora of metals in a number of organic transformations. The extraordinarily high utility of ItBu stems from the large steric volume, i.e. buried volume (% V_{bur}=39.6% for [Au(ItBu)Cl]) provided by the bulky t-Bu group at the N-wingtip. Simultaneously, the electron-donating alkyl groups engender the ligand with strong σ-donation (TEP, 2049 cm⁻¹ for [Rh(ItBu)(CO)₂Cl] and high π-acceptance (⁷⁷Se NMR, δ_{Se} 183 ppm for [Se(ItBu)]), which supersede the values observed for N-aryl NHCs. Overall, this results in a unique NHC scaffold that has become an indispensable part of the synthetic toolbox, while providing direct access to novel reactivity and is now routinely utilized in reaction screening and optimization.

Thus, there is a need in the art for additional improved sterically hindered N-aliphatic NHC ligands, synthetic methods for the preparation thereof, and methods of use thereof. The present disclosure addresses this need.

### BRIEF SUMMARY

The present disclosure relates, in part, to a compound of formula (I):

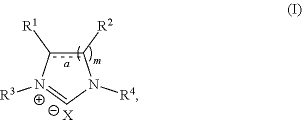

wherein R¹, R², R³, R⁴, X, a, and m are defined within the scope of the present disclosure.

The present disclosure further relates to a compound of formula (II):

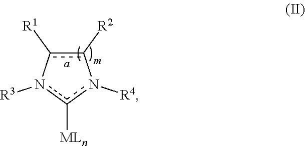

wherein R¹, R², R³, R⁴, M, L, a, m, and n are defined within the scope of the present disclosure.

The present disclosure further provides methods of preparing the compound of formula (I), the method comprising:
contacting a compound of formula (III):

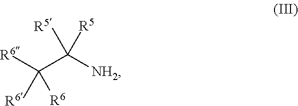

with (CHO)₂ to form a compound of formula (IV):

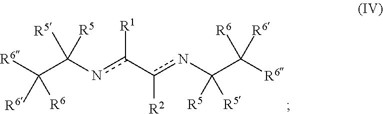

and
cyclizing the compound of formula (IV) to form the compound of formula (I), wherein R¹, R², R⁵, R^{5′}, R⁶, R^{6′}, and R^{6″} are defined within the scope of the present disclosure. In certain embodiments, R³ and R⁴ in the compound of formula (I) are identical.

The present disclosure further provides a method of promoting hydration of an alkyne, the method comprising contacting the alkyne and water in the presence of a compound of formula (II).

The present disclosure further provides a method of promoting a reaction between an alkyne and a borylation reagent, the method comprising contacting the alkyne and the borylation reagent in the presence of a base, a protic solvent or electrophile, and a compound of formula (II).

The present disclosure further provides a method of promoting a reaction between a first reagent and a second reagent, the method comprising contacting the first reagent and the second reagent in the presence of a base and a compound of formula (II), wherein the first reagent and second reagent are defined within the scope of the present disclosure.

The present disclosure further provides a method of promoting a reaction between an aryl bromide and a second reagent, the method comprising contacting the aryl bromide and the second reagent in the presence of a base and a compound of formula (II), wherein the second reagent is defined within the scope of the present disclosure.

### BRIEF DESCRIPTION OF THE FIGURES

The drawings illustrate generally, by way of example, but not by way of limitation, various embodiments of the present application.

FIG. 1A provides the chemical structures of two known sterically hindered N-heterocyclic carbene (NHC) ligands (i.e. ItBu and SItBu) and two sterically hindered NHC ligands of the present disclosure (i.e. ItOct and SItOct). FIG. 1B provides a comparison of the amine precursors used for the synthesis of the NHC ligands provided in FIG. 1A.

FIGS. 2A-2E provide X-ray crystal structures of several NHC catalyst complexes of the present disclosure. FIG. 2A: [Au(ItOct)Cl] front and side views; FIG. 2B: [Cu(ItOct)Cl]; FIG. 2C: [Ag(ItOct)Cl]; FIG. 2D: [Se(ItOct)]; and FIG. 2E: [Pd(ItOct)(3-Cl-py)Cl₂].

FIGS. 3A-3D provide topographical steric maps of several NHC catalyst complexes of the present disclosure, wherein buried volume (% V_{bur}) is provided. FIG. 3A: [Au(ItOct)Cl]; FIG. 3B: [Cu(ItOct)Cl]; FIG. 3C: [Ag(ItOct)Cl]; and [Pd(ItOct)(3-Cl-py)Cl₂].

FIGS. 4A-4F provide reaction schemes demonstrating the activity of ItOct NHC catalyst complexes, as compared to ItBu NHC catalyst complexes in catalytic reactions. FIG. 4A: Au-catalyzed alkyne hydration; FIG. 4B: Cu-catalyzed alkyne hydroboration; FIG. 4C: Ag-catalyzed alkyne hydroboration; FIG. 4D: Cu-catalyzed C—O coupling; FIG. 4E: Pd-catalyzed C—C coupling; and FIG. 4F: Pd-catalyzed C—N coupling.

FIG. 5 provides a graphical representation of the highest occupied molecular orbital (HOMO) and lowest unoccupied molecular orbital (LUMO) of ItOct, as well as the respective energies thereof, calculated at B3LYP 6-311++g(d,p).

FIG. 6 provides a plot of % V_{bur} vs. Charton parameter in [Au(NHC)Cl] complexes, demonstrating that ItOct is the most sterically-demanding N-alkyl-NHC reported.

### DETAILED DESCRIPTION

The present disclosure relates in one aspect to sterically hindered N-aliphatic N-heterocyclic carbene ligands and metal catalyst complexes thereof. The present disclosure further relates to the synthesis, structural characterization, and catalytic activity of the NHC catalyst complexes described herein.

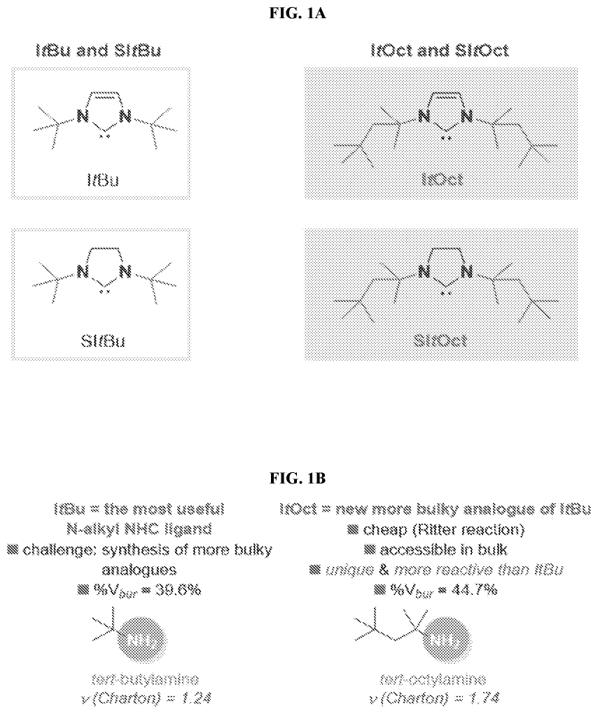

Exemplary sterically hindered N-alkyl NHC ligands of the present disclosure include, but are not limited to ItOct (1,3-bis(1,1,3,3-tetramethylbutyl)imidazole-2-ylidene) and SItOct (1,3-bis(1,1,3,3-tetramethylbutyl)-4,5-dihydro-imidazole-2-ylidene), a saturated homolog thereof (FIGS. 1A-1B). Herein, it is demonstrated that exchange of ItBu for ItOct results in significantly higher steric volume (% V_{bur}=44.7%), which matches values observed for the archetypal N-aryl NHCs (IPr, % V_{bur}=45.4%; IMes, % V_{bur}=36.5% for [Au(NHC)Cl] complexes), while retaining the electronic properties of the N-alkyl ligand, such as strong σ-donation and π-acceptance inherent to N-alkyl NHCs. Large scale synthesis, beneficial effects in catalysis, and insights into the structure and electronic properties of the carbene center of the presently disclosed NHC catalysts are described herein. Considering the key importance and immense utility of ItBu in organic synthesis, the new class of sterically hindered N-aliphatic NHCs described herein finds use in a variety of applications, including the areas of synthesis, catalysis, and medicinal chemistry.

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “anionic ligand” as used herein, refers to a ligand having a net negative charge prior to association with, or after dissociation from, a metal center. Non-limiting examples of anionic ligands include, F, Cl, Br, I, and OMe.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aralkynyl” as used herein refers to alkynyl groups as defined herein in which a hydrogen or carbon bond of an alkynyl group is replaced with a bond to an aryl group as defined herein. Representative aralkynyl groups include, but are not limited to, phenylacetylene and naphthylacetylene.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2, 3, 4, 5, or 6-positions of the phenyl ring, or a naphthyl group substituted at any one or more of 2- to 8-positions thereof.

The term “arylhydroxy” as used herein refers to an aryl group, as defined elsewhere herein, substituted with at least one hydroxyl moiety at any position of at least one aryl group. Non-limiting examples of arylhydroxy compounds include phenol and 1-naphthol.

The term “borylation reagent” as used herein refers to any of a number of electrophilic boron containing species, including, but not limited to boranes, diboranes, boronic acids (RB(OH)₂), boronic esters (RB(OR)₂), or diboronic esters ((OR)₂B—B(OR²)), which, either independently or in the presence of additional reagents and/or catalysts, are suitable to react with an alkene or alkyne to provide an addition product.

The term “B₂(pin)₂” refers to bis(pinacolato)diboron.

The term “counter anion” as used herein refers to a negatively charged ion that accompanies a cationic species (i.e. positively charged ion) in order to maintain electric neutrality. For example, the chloride ion (Cl⁻) is the counter anion to sodium (Na⁺) in NaCl. Non-limiting examples of counter anions include F⁻, Cl⁻, Br⁻, I⁻, N₃⁻, F₃CS(═O)₂O⁻ (OTf), (F₃CS(═O)₂)₂N⁻ (NTf₂), F₃CC(═O)O⁻ (TFA), BF₄⁻, and PF₆⁻.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2, 2,3, 2,4-2,5- or 2,6-disubstituted cyclohexyl groups or mono, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “electrophile” as used herein refers to a chemical species that forms a bond with a nucleophile by accepting an electron pair in a chemical reaction (e.g. S_{N}1, S_{N}2, and carbonyl [1,2]-addition. Non-limiting examples of electrophiles include, but not limited to, alkyl halides (e.g. MeI), benzyl halides (e.g. BnBr), dihalides (e.g. Br₂), aldehydes (e.g. Ph-CHO), acyl halides (e.g. Ph(C═O)Cl), N-electrophiles (e.g. RC(═O)ONR²), and O-electrophiles (e.g. RC(═O)O₂R).

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxybutyl 4,5-epoxypentyl, 2,3-epoxypropoxy, epoxypropoxypropyl, 2-glycidoxyethyl, 3-glycidoxypropyl, 4-glycidoxybutyl, 2-(glycidoxycarbonyl)propyl, 3-(3,4-epoxycylohexyl)propyl, 2-(3,4-epoxycyclohexyl)ethyl, 2-(2,3-epoxycylopentyl)ethyl, 2(4-methyl-3,4-epoxycyclohexyl)propyl, 2-(3,4-epoxy-3-methylcylohexyl)-2-methylethyl, and 5,6-epoxyhexyl.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaralkynyl” as used herein refers to alkynyl groups as defined herein in which a hydrogen or carbon bond of an alkynyl group is replaced with a bond to a heteroaryl group as defined herein. Representative aralkynyl groups include, but are not limited to, 2-ethynylpyridine and 2-ethynylthiophene.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C2-heteroaryl can be a 5-ring with two carbon atoms and three heteroatoms, a 6-ring with two carbon atoms and four heteroatoms and so forth. Likewise a C4-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl) , indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3- pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylalkyl” as used herein refers to alkyl groups as defined herein in which a hydrogen or carbon bond of an alkyl group is replaced with a bond to a heteroaryl group as defined herein.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C2-heterocyclyl can be a 5-ring with two carbon atoms and three heteroatoms, a 6-ring with two carbon atoms and four heteroatoms and so forth. Likewise a C4-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2, 3, 4, 5, or 6-substituted, or disubstituted with groups such as those listed herein.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “Lewis acid” as used herein refers to a chemical species that possesses an empty orbital which is capable of accepting electrons from a Lewis base.

The term “monovalent” as used herein refers to a substituent connecting via a single bond to a substituted molecule. When a substituent is monovalent, such as, for example, F or Cl, it is bonded to the atom it is substituting by a single bond.

The term “neutral ligand” as used herein, refers to a ligand having no net charge prior to association with, or after dissociation from, a metal center. Non-limiting examples of neutral ligands include, alkene, CO, PPh3, and pyridyl ligands, wherein coordination occurs via the nitrogen lone pair of the pyridyl group.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(=NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “room temperature” as used herein refers to a temperature of about 15° C. to 28° C.

The term “solvent” as used herein refers to a liquid that can dissolve a solid, liquid, or gas. Non-limiting examples of solvents are silicones, organic compounds, water, alcohols, ionic liquids, and supercritical fluids.

The term “standard temperature and pressure” as used herein refers to 20° C. and 101 kPa.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” or as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

### Compounds

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base- protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference.

The compounds of the present disclosure may be prepared as described herein, using synthetic methods known by those skilled in the art. The following examples illustrate non- limiting embodiments of the compound(s) described herein and their preparation.

In one aspect, the present disclosure provides a compound of formula (I):

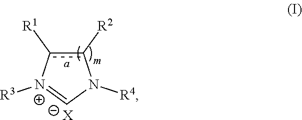

wherein:
X is a counter anion;
R¹ and R² are each independently selected from the group consisting of H, optionally substituted C₁-C₆ alkyl, optionally substituted C₂-C₆ alkenyl, optionally substituted benzyl, optionally substituted phenyl, and optionally substituted naphthyl, and optionally substituted C₄-C₁₀ heteroaryl, or
   R¹ and R² may combine with the ring to which they are attached to form C₅-C₁₂ cycloalkyl, C₆-C₁₀ aryl, or C₄-C₁₀ heteroaryl,
   wherein each optional substituent in R¹ and R² is independently at least one substituent selected from the group consisting of C₁-C₃ alkyl, C₂-C₆ alkenyl, phenyl, naphthyl, C₄-C₁₀ heteroaryl, N(R^{a})(R^{b}), OR^{b}, CN, CF₃, OCF₃, C(═O)R^{b}, N(R)S(═O)₂R^{b}, C(═O)N(R^{a})(R^{b}), and C(═O)OR^{b};

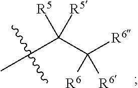

R³ and R⁴ are each independently
R⁵ and R^{5′} are each independently selected from the group consisting of C₁-C₆ alkyl, C₃-C₁₂ cycloalkyl, C₂-C₁₀ heterocycloalkyl, C₆-C₁₀ aryl, C₄-C₁₀ heteroaryl, halogen, OR^{b}, N(R^{a})(R^{b}), SR^{b}, and Si(R^{b})₃,
   wherein R⁵ and R^{5′} may combine with the carbon atom to which they are bound to form one selected from the group consisting of C₃-C₈ cycloalkyl and C₂-C₇ heterocycloalkyl;
R⁶, R^{6′}, and R^{6″} are each independently selected from the group consisting of H, C₁-C₆ alkyl, C₃-C₁₂ cycloalkyl, C₂-C₁₀ heterocycloalkyl, C₆-C₁₀ aryl, C₄-C₁₀ heteroaryl, halogen, OR^{b}, N(R^{a})(R^{b}), SR^{b}, and Si(R^{b})₃,
   wherein at least two of R⁶, R^{6′}, and R^{6″} may combine with the carbon atom to which they are bound to form one selected from the group consisting of C₃-C₁₂ cycloalkyl, C₂-C₁₀ heterocycloalkyl, C₅-C₁₂ fused cycloalkyl, C₄-C₁₀ fused heterocycloalkyl, C₅-C₁₂ bridged cycloalkyl, and C₄-C₁₀ bridged heterocycloalkyl
   wherein any one of R⁶, R^{6′}, and R^{6″}, and any one of R⁵ and R^{5′}, may combine with the carbon atoms to which they are bound to form one selected from the group consisting of C₄-C₁₂ cycloalkyl and C₃-C₁₀ heterocycloalkyl, and
   wherein if R⁵ and R^{5′} are Me, and two of R⁶, R^{6′}, and R^{6″} are H, then the other one of R⁶, R^{6′}, and R^{6″}is not H or Me;
   wherein if R⁵ and R^{5′} are Me, two of R⁶, R^{6′}, and R^{6″} are H, and one of R⁶, R^{6′}, and R^{6″} is t-Bu, then M is not BF4;
bond a is a single or double bond;
each occurrence of R^{a} is independently H or C₁-C₃ alkyl;
each occurrence of R^{b} is independently selected from the group consisting of H, C₁-C₆ alkyl, C₁-C₃ haloalkyl, C₂-C₆ alkenyl, benzyl, naphthyl, C₄-C₁₀ heteroaryl, and phenyl optionally substituted with at least one substituent selected from the group consisting of C₁-C₃ alkyl and halogen; and
m is either 1 or 2.

In certain embodiments, X is selected from the group consisting of H, halogen, OS(═O)₂R^{b}, OC(═O)R^{b}, N(C(═O)R^{b})₂, tetracoordinate boronate, and hexacoordinate phosphorus. In certain embodiments, X is Cl. In certain embodiments, X is BF₄.

In certain embodiments, at least one of R⁵ and R^{5′} is Me. In certain embodiments, at least one of R⁵ and R^{5′} is Et.

In certain embodiments, two of R⁶, R^{6′}, and R^{6″} are H. In certain embodiments, at least one of R⁶, R^{6′}, and R^{6″} is tert-butyl. In certain embodiments, at least one of R⁶, R^{6′}, and R^{6″} is Me.

In certain embodiments, at least one of R¹ and R² is H or Me. In certain embodiments, R¹ and R² are identical.

In certain embodiments, at least one of R³ and R⁴ is

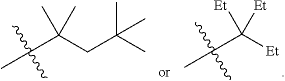

In certain embodiments, R³ and R⁴ are identical.

In certain embodiments, bond a is a single bond. In certain embodiments, bond a is a double bond.

In certain embodiments, m is 1.

In certain embodiments, the compound is selected from the group consisting of:
1,3-bis(2,4,4-trimethylpentan-2-yl)-1H-imidazol-3-ium chloride;
1,3-bis(2,4,4-trimethylpentan-2-yl)-1H-imidazol-3-ium tetrafluoroborate;
1,3-bis(2,4,4-trimethylpentan-2-yl)-4,5-dihydro-1H-imidazol-3-ium chloride;
1,3-bis(2,4,4-trimethylpentan-2-yl)-1H-benzo[d]imidazol-3-ium chloride;
1,3-bis(3-ethylpentan-3-yl)-1H-imidazol-3-ium chloride; and
1,3-bis(3-ethylpentan-3-yl)-4,5-dihydro-1H-imidazolium chloride.

In another aspect, the present disclosure provides a compound of formula (II):

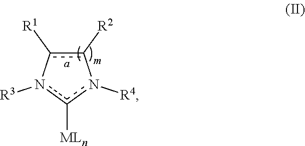

wherein:
M is a transition metal;
L is a ligand of M, wherein each occurrence of L can be the same or different;
R¹ and R² are each independently selected from the group consisting of H, optionally substituted C₁-C₆ alkyl, optionally substituted C₂-C₆ alkenyl, optionally substituted benzyl, optionally substituted phenyl, and optionally substituted naphthyl, and optionally substituted C₄-C₁₀ heteroaryl, or
   R¹ and R² may combine with the ring to which they are attached to form a C₅-C₁₂ cycloalkyl, C₆-C₁₀ aryl, or C₄-C₁₀ heteroaryl,
   wherein each optional substituent in R¹ and R² is independently at least one substituent selected from the group consisting of C₁-C₃ alkyl, C₂-C₆ alkenyl, phenyl, naphthyl, C₄-C₁₀ heteroaryl, N(R^{a})(R^{b}), OR^{b}, CN, CF₃, OCF₃, C(═O)R^{b}, N(R)S(═O)₂R^{b}, C(═O)N(R^{a})(R^{b}), and C(═O)OR^{b};
R³ and R⁴ are each independently

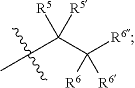

R⁵ and R^{5′} are each independently selected from the group consisting of C₁-C₆ alkyl, C₃-C₁₂ cycloalkyl, C₂-C₁₀ heterocycloalkyl, C₆-C₁₀ aryl, C₄-C₁₀ heteroaryl, halogen, OR^{b}, N(R^{a})(R^{b}), SR^{b}, and Si(R^{b})₃,
   wherein R⁵ and R^{5′} may combine with the carbon atom to which they are bound to form one selected from the group consisting of C₃-C₈ cycloalkyl and C₂-C₇ heterocycloalkyl;
R⁶, R^{6′}, and R^{6″} are each independently selected from the group consisting of H, C₁-C₆ alkyl, C₃-C₁₂ cycloalkyl, C₂-C₁₀ heterocycloalkyl, C₆-C₁₀ aryl, C₄-C₁₀ heteroaryl, halogen, OR^{b}, N(R^{a})(R^{b}), SR^{b}, and Si(R^{b})₃,
   wherein at least two of R⁶, R^{6′}, and R^{6″} may combine with the carbon atom to which they are bound to form one selected from the group consisting of C₃-C₁₂ cycloalkyl, C₂-C₁₀ heterocycloalkyl, C₅-C₁₂ fused cycloalkyl, C₄-C₁₀ fused heterocycloalkyl, C₅-C₁₂ bridged cycloalkyl, and C₄-C₁₀ bridged heterocycloalkyl,
   wherein any one of R⁶, R^{6′}, and R^{6″}, and any one of R⁵ and R^{5′}, may combine with the carbon atoms to which they are bound to form one selected from the group consisting of C₄-C₁₂ cycloalkyl and C₃-C₁₀ heterocycloalkyl, and
   wherein if R⁵ and R^{5′} are Me, and two of R⁶, R^{6′}, and R^{6″} are H, then the other one of R⁶, R^{6′}, and R^{6″} is not H or Me;
each occurrence of [symbol] is a single or double bond,
   wherein no more than one [symbol] bonding a C atom and a N atom is a double bond;
each occurrence of R^{a} is independently H or C₁-C₃ alkyl;
each occurrence of R^{b} is independently selected from the group consisting of H, C₁-C₆ alkyl, C₁-C₃ haloalkyl, C₂-C₆ alkenyl, benzyl, naphthyl, C₄-C₁₀ heteroaryl, and phenyl optionally substituted with at least one substituent selected from the group consisting of C₁-C₃ alkyl and halogen;
n is an integer which is selected from the group consisting of 1, 2, 3, 4, and 5; and
m is an integer which is either 1 or 2.

In certain embodiments, M is Cu. In certain embodiments, M is Ag. In certain embodiments, M is Au. In certain embodiments, M is Pd. In certain embodiments, M is Ni. In certain embodiments, M is Pt. In certain embodiments, M is Co. In certain embodiments, M is Rh. In certain embodiments, M is Ir. In certain embodiments, M is Fe. In certain embodiments, M is Ru. In certain embodiments, M is Os.

In certain embodiments, L is an anionic ligand. In certain embodiments, L is a neutral ligand. In certain embodiments, the anionic ligand is H. In certain embodiments, the anionic ligand is OS(═O)₂R^{b}. In certain embodiments, the anionic ligand is OC(═O)R^{b}. In certain embodiments, the anionic ligand is N(C(═O)R^{b})₂. In certain embodiments, the anionic ligand is a halogen. In certain embodiments, the anionic ligand is C₆-C₁₀ aryl. In certain embodiments, the anionic ligand is C₄-C₁₀ heteroaryl. In certain embodiments, the anionic ligand is Cl. In certain embodiments, the neutral ligand is pyridyl optionally substituted with at least one halogen. In certain embodiments, the neutral ligand is

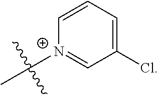 [SMILES: *[n+]1cccc(Cl)c1]

In certain embodiments, the compound is selected from the group consisting of:
1,3-bis(2,4,4-trimethylpentan-2-yl)-2,3-dihydro-1H-imidazol-2-ylidene gold(I) chloride;
1,3-bis(2,4,4-trimethylpentan-2-yl)-2,3-dihydro-1H-imidazol-2-ylidene copper(I) chloride;
1,3-bis(2,4,4-trimethylpentan-2-yl)-2,3-dihydro-1H-imidazol-2-ylidene silver(I) chloride;
1,3-bis(2,4,4-trimethylpentan-2-yl)-2,3-dihydro-1H-imidazol-2-ylidene palladium(II)(allyl) chloride;
3-chloropyridine [1,3-bis(2,4,4-trimethylpentan-2-yl)-2,3-dihydro-1H-imidazol-2-ylidene] palladium(II) dichloride;
1,3-bis(2,4,4-trimethylpentan-2-yl)imidazolidin-2-ylidene gold(I) chloride;
1,3-bis(2,4,4-trimethylpentan-2-yl)imidazolidin-2-ylidene palladium(II) (allyl) chloride;
1,3-bis(2,4,4-trimethylpentan-2-yl)benzimidazolidin-2-ylidene gold(I) chloride;
1,3-bis(2,4,4-trimethylpentan-2-yl)benzimidazolidin-2-ylidene copper(I) chloride;
3-chloropyridine [1,3-bis(2,4,4-trimethylpentan-2-yl)-benzimidazol-2-ylidene] palladium(II) dichloride
allyl [1,3-bis(2,4,4-trimethylpentan-2-yl)benzimidazolidin-2-ylidene] palladium(II) chloride;
1,3-bis(3-ethylpentan-3-yl)-1H-imidazol-2-ylidene gold(I) chloride;
1,3-bis(3-ethylpentan-3-yl)-1H-imidazol-2-ylidene copper(I) chloride;
1,3-bis(3-ethylpentan-3-yl)-1H-imidazol-2-ylidene silver(I) chloride;
3-chloropyridine [1,3-bis(3-ethylpentan-3-yl)-1H-imidazol-2-ylidene] palladium(II) dichloride;
1,3-bis(3-ethylpentan-3-yl)-4,5-dihydro-H-imidazol-2-ylidene gold(I) chloride; and
1,3-bis(3-ethylpentan-3-yl)-4,5-dihydro-1H-imidazol-2-ylidene copper(I) chloride.

TABLE 1

| Structure | Name |
|---|---|
| 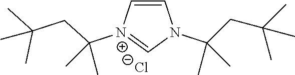 [SMILES: CC(C)(C)CC(C)(C)n1cc[n+](C(C)(C)CC(C)(C)C)c1 ; [Cl-]] | 1,3-bis(2,4,4-trimethylpentan-2-yl)-1H-imidazol-3-ium chloride |
| 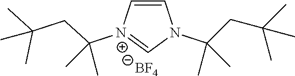 | 1,3-bis(2,4,4-trimethylpentan-2-yl)-1H-imidazol-3-ium tetrafluoroborate |
| 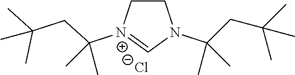 [SMILES: CC(C)(C)CC(C)(C)N1C=[N+](C(C)(C)CC(C)(C)C)CC1 ; [Cl-]] | 1,3-bis(2,4,4-trimethylpentan-2-yl)-4,5-dihydro-1H- imidazol-3-ium chloride |
| 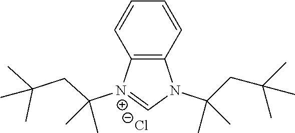 [SMILES: CC(C)(C)CC(C)(C)n1c[n+](C(C)(C)CC(C)(C)C)c2ccccc21 ; [Cl-]] | 1,3-bis(2,4,4-trimethylpentan-2-yl)-1H-benzo[d]imidazol-3- ium chloride |
| 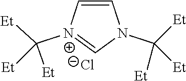 [SMILES: CCC(CC)(CC)n1cc[n+](C(CC)(CC)CC)c1 ; [Cl-]] | 1,3-bis(3-ethylpentan-3-yl)-1H-imidazol-3-ium chloride |
| 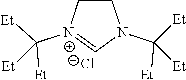 [SMILES: CCC(CC)(CC)N1C=[N+](C(CC)(CC)CC)CC1 ; [Cl-]] | 1,3-bis(3-ethylpentan-3-yl)-4,5-dihydro-1H-imidazolium |
| 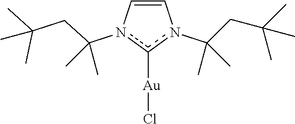 | 1,3-bis(2,4,4-trimethylpentan-2-yl)-2,3-dihydro-1H- imidazol-2-ylidene gold(I) chloride |
| 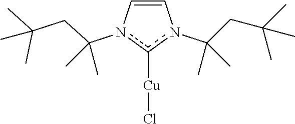 | 1,3-bis(2,4,4-trimethylpentan-2-yl)-2,3-dihydro-1H- imidazol-2-ylidene copper(I) chloride |
| 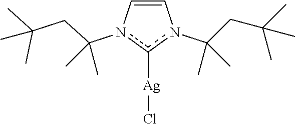 | 1,3-bis(2,4,4-trimethylpentan-2-yl)-2,3-dihydro-1H- imidazol-2-ylidene silver(I) chloride |
| 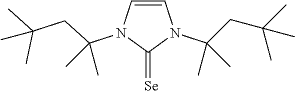 [SMILES: CC(C)(C)CC(C)(C)n1ccn(C(C)(C)CC(C)(C)C)c1=[Se]] | 1,3-bis(2,4,4-trimethylpentan-2-yl)-2,3-dihydro-1H- imidazol-2-ylidene selenide |
| 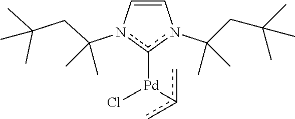 | 1,3-bis(2,4,4-trimethylpentan-2-yl)-2,3-dihydro-1H- imidazol-2-ylidene palladium(II) (allyl) chloride |
| 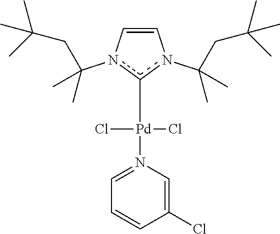 | 3-chloropyridine [1,3-bis(2,4,4-trimethylpentan-2-yl)-2,3- dihydro-1H-imidazol-2-ylidene] palladium(II) dichloride |
| 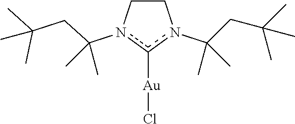 | 1,3-bis(2,4,4-trimethylpentan-2-yl)imidazolidin-2-ylidene gold(I) chloride |
| 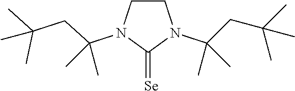 [SMILES: CC(C)(C)CC(C)(C)N1CCN(C(C)(C)CC(C)(C)C)C1=[Se]] | 1,3-bis(2,4,4-trimethylpentan-2-yl)imidazolidin-2-ylidene selenide |
| 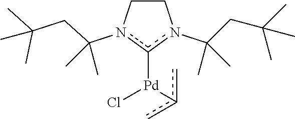 | 1,3-bis(2,4,4-trimethylpentan-2-yl)imidazolidin-2-ylidene palladium(II) (allyl) chloride |
| 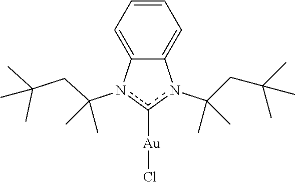 | 1,3-bis(2,4,4-trimethylpentan-2-yl)benzimidazolidin-2- ylidene gold(I) chloride |
| 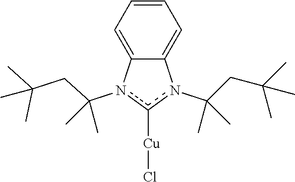 | 1,3-bis(2,4,4-trimethylpentan-2-yl)benzimidazolidin-2- ylidene copper(I) chloride |
| 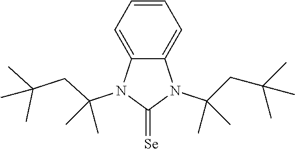 [SMILES: CC(C)(C)CC(C)(C)n1c(=[Se])n(C(C)(C)CC(C)(C)C)c2ccccc21] | 1,3-bis(2,4,4-trimethylpentan-2-yl)-1,3-dihydro-2H- benzo[d]imidazole-2-selenone |
| 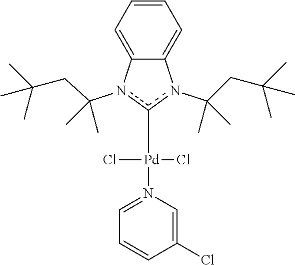 | 3-chloropyridine [1,3-bis(2,4,4-trimethylpentan-2-yl)- benzimidazol-2-ylidene] palladium(II) dichloride |
| 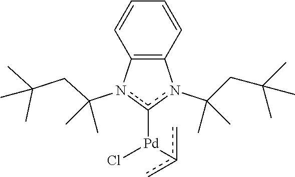 | Allyl [1,3-bis(2,4,4-trimethylpentan-2-yl)benzimidazolidin- 2-ylidene] palladium(II) chloride |
| 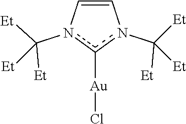 | 1,3-bis(3-ethylpentan-3-yl)-1H-imidazol-2-ylidene gold(I) chloride |
| 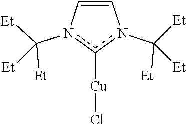 | 1,3-bis(3-ethylpentan-3-yl)-1H-imidazol-2-ylidene copper(I) chloride |
| 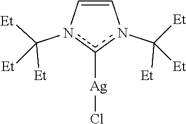 | 1,3-bis(3-ethylpentan-3-yl)-1H-imidazol-2-ylidene silver(I) chloride |
| 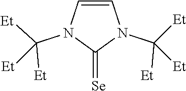 [SMILES: CCC(CC)(CC)n1ccn(C(CC)(CC)CC)c1=[Se]] | 1,3-bis(3-ethylpentan-3-yl)-1,3-dihydro-2H-imidazole-2- selenone |
| 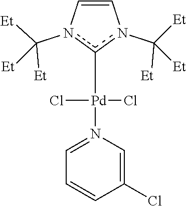 | 3-chloropyridine [1,3-bis(3-ethylpentan-3-yl)-1H-imidazol- 2-ylidene] palladium(II) dichloride |
| 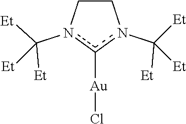 | 1,3-bis(3-ethylpentan-3-yl)-4,5-dihydro-1H-imidazol-2- ylidene gold(I) chloride |
| 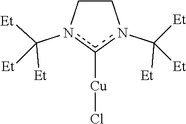 | 1,3-bis(3-ethylpentan-3-yl)-4,5-dihydro-1H-imidazol-2- ylidene copper(I) chloride |
| 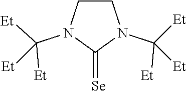 [SMILES: CCC(CC)(CC)N1CCN(C(CC)(CC)CC)C1=[Se]] | 1,3-bis(3-ethylpentan-3-yl)imidazolidine-2-selenone |

### Methods

In certain embodiments, the present disclosure provides a method of preparing the compound of formula (I), the method comprising: contacting a compound of formula (III):

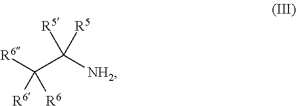

with (CHO)₂ to form a compound of formula (IV):

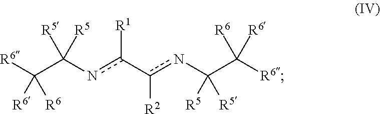

and cyclizing the compound of formula (IV) to form the compound of formula (I).

In certain embodiments, the compound of formula (IV) is a compound of formula (IVa):

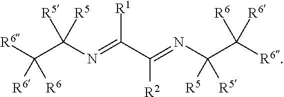

In certain embodiments, the compound of formula (IV) is treated with NaBH4 to form a diamine compound of formula (IVb):

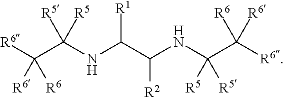

In certain embodiments, R³ and R⁴ in the compound of formula (I) are identical.

In certain embodiments, the cyclizing step comprises treatment of the compound of formula (IV) with HCl and either (CH₂O)ₙ at a temperature of about 60° C. or HC(OEt)₃ and HCO₂H at a temperature of about 125° C.

In another aspect, the present disclosure provides a method of promoting hydration of an alkyne, the method comprising contacting the alkyne and water in the presence of a NHC catalyst of the present disclosure.

In certain embodiments, the alkyne is selected from the group consisting of optionally substituted C₂-C₁₂ alkynyl, optionally substituted C₆-C₁₀ aralkynyl, and optionally substituted C₄-C₁₀ heteroaralkynyl, wherein each optional substituent is at least one selected from the group consisting of C₁-C₆ alkoxy, C₁-C₆ haloalkoxy, C₁-C₆ haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, halogen, OH, NH₂, NH(C₁-C₆ alkyl), N(C₁-C₆ alkyl)₂, CN, NO₂, CHO, C(═O)OH, C(═O)O(C₁-C₆ alkyl), C(═O)NH₂, C(═O)NH(C₁-C₆ alkyl), and C(═O)N(C₁-C₆ alkyl)₂.

In certain embodiments, the method comprises a Lewis acid. In certain embodiments, the Lewis acid is selected from the group consisting of AgNTf₂, AgOAc, AgOTf, NaBArF, KB(C₆F₅)₄, and AgSbF₆.

In certain embodiments, the contacting occurs in the presence of a solvent. In certain embodiments, the solvent is 1,4-dioxane.

In certain embodiments, the contacting occurs at a temperature of about 60, 70, 80, 90, 100, 110, 120, 130, or about 140° C.

In certain embodiments, the NHC catalyst of the present disclosure is present in an amount ranging from about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or about 2.0 mol %.

In another aspect, the present disclosure provides a method of promoting a reaction between an alkyne and a borylation reagent, the method comprising contacting the alkyne and the borylation reagent in the presence of a base, a protic solvent or electrophile, and a NHC catalyst of the present disclosure.

In certain embodiments, the alkyne is selected from the group consisting of optionally substituted C₂-C₁₂ alkynyl, optionally substituted C₆-C₁₀ aralkynyl, and optionally substituted C₄-C₁₀ heteroaralkynyl, wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, halogen, OH, NH₂, NH(C₁-C₆ alkyl), N(C₁-C₆ alkyl)₂, CN, NO₂, CHO, C(═O)OH, C(═O)O(C₁-C₆ alkyl), C(═O)NH₂, C(═O)NH(C₁-C₆ alkyl), and C(═O)N(C₁-C₆ alkyl)₂.

In certain embodiments, the borylation reagent is a diboronic ester. In certain embodiments, the diboronic ester is B₂(pin)₂. In certain embodiments, the NHC catalyst of the present disclosure is present in an amount of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or about 3.0 mol %.

In certain embodiments, the protic solvent is MeOH.

In certain embodiments, the base is KOt-Bu.

In certain embodiments, the electrophile is selected from the group consisting of C₁-C₁₂ haloalkyl, C₆-C₁₂ haloaralkyl, C₆-C₁₀ aryl halide, C₄-C₁₀ heteroaryl halide, C₁-C₁₂ alkyl-(C═O)Cl, C₆-C₁₀ aryl-(C═O)Cl, C₄-C₁₀ heteroaryl-(C═O)Cl, and CO₂. In certain embodiments, the electrophile is MeI.

In certain embodiments, the contacting occurs at a temperature ranging from about 20 to about 70° C.

In another aspect, the present disclosure provides a method of promoting a reaction between a first reagent and a second reagent, the method comprising contacting the first reagent and the second reagent in the presence of a base and a NHC catalyst of the present disclosure.

In certain embodiments, the first reagent is selected from the group consisting of optionally substituted C₆-C₁₀ aryl iodide and optionally substituted C₄-C₁₀ heteroaryl iodide, wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, N(C₁-C₆ alkyl)₂, CN, NO₂, C(═O)NH₂, C(═O)NH(C₁-C₆ alkyl), and C(═O)N(C₁-C₆ alkyl)₂.

In certain embodiments, the second reagent is a optionally substituted C₆-C₁₀ hydroxyaryl, wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, N(C₁-C₆ alkyl)₂, CN, NO₂, C(═O)NH₂, C(═O)NH(C₁-C₆ alkyl), and C(═O)N(C₁-C₆ alkyl)₂.

In certain embodiments, the base is Cs₂CO₃.

In certain embodiments, the contacting occurs in the presence of a solvent. In certain embodiments, the solvent is DMF.

In certain embodiments, the NHC catalyst of the present disclosure is present in an amount of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or about 3.0 mol %.

In certain embodiments, the contacting occurs at a temperature of about 120, 130, 140, 150, 160, 170, or about 180° C.

In another aspect, the present disclosure provides a method of promoting a reaction between an aryl bromide and a second reagent, the method comprising contacting the aryl bromide and the second reagent in the presence of a base and a NHC catalyst of the present disclosure.

In certain embodiments, the aryl bromide is selected from the group consisting of optionally substituted C₆-C₁₀ aryl bromide and optionally substituted C₄-C₁₀ heteroaryl bromide, wherein each optional substituent is at least one selected the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, N(C₁-C₆ alkyl)₂, CN, NO₂, C(═O)NH₂, C(═O)NH(C₁-C₆ alkyl), and C(═O)N(C₁-C₆ alkyl)₂.

In certain embodiments, the NHC catalyst of the present disclosure is present in an amount of about 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, or about 6.0 mol %.

In certain embodiments, the contacting occurs at a temperature of about 50, 60, 70, 80, 90, 100, 110, 120, 130, 140, or about 150° C.

In certain embodiments, the base is selected from the group consisting of K₂CO₃ and KOt-Bu.

In certain embodiments, the second reagent is a secondary amine.

In certain embodiments, the secondary amine is selected from the group consisting of optionally substituted C₄-C₁₂ heterocycloalkyl and optionally substituted NH(C₁-C₆ alkyl)₂, wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, N(C₁-C₆ alkyl)₂, CN, NO₂, C(═O)NH₂, C(═O)NH(C₁-C₆ alkyl), and C(═O)N(C₁-C₆ alkyl)₂.

In certain embodiments, the contacting occurs in the presence of a protic solvent. In certain embodiments, the protic solvent is i-PrOH.

In certain embodiments, the second reagent is selected from the group consisting of optionally substituted C₆-C₁₀ aryl boronic acid and optionally substituted C₄-C₁₀ heteroaryl boronic acid, wherein each optional substituent is at least one selected the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, N(C₁-C₆ alkyl)₂, CN, NO₂, C(═O)NH₂, C(═O)NH(C₁-C₆ alkyl), and C(═O)N(C₁-C₆ alkyl)₂.

### EXAMPLES

Various embodiments of the present application can be better understood by reference to the following Examples which are offered by way of illustration. The scope of the present application is not limited to the Examples given herein.

### Example 1: Synthesis of NHC Catalyst Precursors

Studies were initiated by developing a flexible and robust synthesis of ItOct (1) imidazolium precursor using the readily available tert-octylamine as the starting material (Scheme 1). Tert-octylamine is considerably cheaper than other bulky amines and readily available on kg scale by the Ritter reaction of the isomeric 2,2,4-trimethylpentenes. As shown in Scheme 1, the synthesis of ItOct proceeds in a cost-effective and straightforward manner. Thus, condensation of tert-octylamine (1-1) with glyoxal (1-2) at room temperature and cyclization of the diimine (1-3) using a combination of HCl/(CH₂O)n in toluene at 60° C. delivered the desired ItOct as a HCl salt (1-Cl) after simple filtration, allowing for a routine preparation of gram quantities of the product (step 1: 98% yield, 61 mmol scale; step 2: 73% yield, 10 mmol scale).

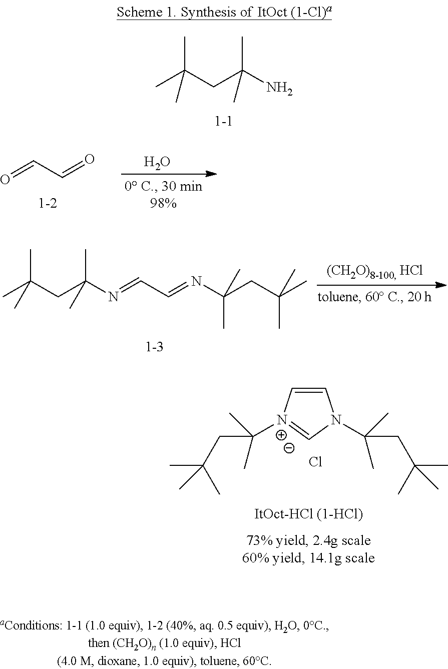

The synthesis of ItOct as HBF₄ salt (1-HBF₄) was optimized to proceed in 82% yield (Scheme 2), while a one-step procedure by combination of tert-octylamine, HBF₄/(CH₂O)ₙ and glyoxal in 58% yield on 2.2 g scale and 82% yield on 1.55 g scale.

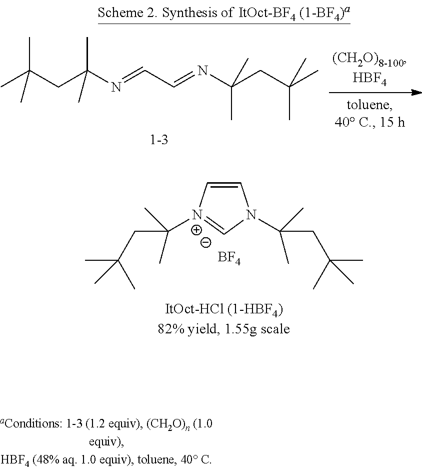

Similarly, the synthesis of SItOct as HCl salt (2-HCl) was accomplished by the sequence of reduction of diamine 1-3 to diamine 3-1 using NaBH4 in MeOH and THF at room temperature (91% yield, 18 mmol scale) and cyclization to the imidazolinium SItOct salt (2-HCl) using a combination of HC(OEt)₃/HCO₂H at 125° C. (77% yield, 5 mmol scale) (Scheme 3). The synthesis is highly practical and allows for the isolation of ItOct·HCl, ItOct·HBF₄ and SItOct·HCl by simple filtration and recrystallization from the reaction mixtures.

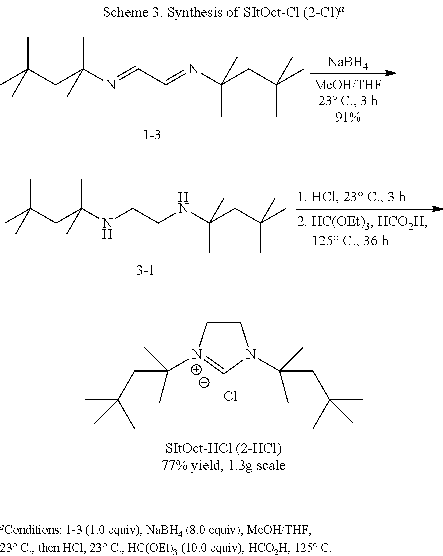

### Synthesis of NHC Catalyst Precursors

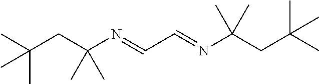 [SMILES: CC(C)(C)CC(C)(C)/N=C/C=N/C(C)(C)CC(C)(C)C]

(1E,2E)-N1,N2-bis(2,4,4-trimethylpentan-2-yl)ethane-1,2-diimine (1-3). A 250 mL round-bottomed flask equipped with a stir bar was charged with glyoxal (aq., 40% w/w, 10.0 mL, 87.2 mmol) and water (40.0 mL) and placed in a water bath. t-Octylamine (20.0 mL, 124.5 mmol) was added dropwise over 60 s with continuous stirring at room temperature. After stirring for 5 min, the reaction mixture was cooled down to 0° C., and stirred for 30 min at 0° C. After the indicated time, the resulting precipitate was filtered, washed with an ice-cold water and dried under vacuum to obtain the title product as white solid in (17.1 g, 98% yield). Mp=45-46° C. ¹H NMR (500 MHz, CDCl₃) δ7.90 (s, 2H), 1.66 (s, 4H), 1.25 (s, 12H), 0.89 (s, 18H). ¹³C NMR (126 MHz, CDCl₃) δ157.93, 62.27, 56.17, 32.41, 31.98, 29.71. IR (KBr, v, cm⁻¹): 2956, 2899, 2868, 1621, 1463, 1361, 1355, 1216. HRMS (ESI) m/z: [M+H]⁺ Calcd for C₁₈H₃₇N₂ 281.2951; Found 281.2954.

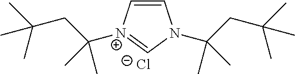 [SMILES: CC(C)(C)CC(C)(C)n1cc[n+](C(C)(C)CC(C)(C)C)c1 ; [Cl-]]

1,3-bis(2,4,4-trimethylpentan-2-yl)-1H-imidazol-3-ium chloride (1-Cl). A mixture of paraformaldehyde (305.0 mg, 10.0 mmol) and HCl (4.0 N, dioxane, 2.5 mL, 10.0 mmol) was stirred for 1 h at room temperature. After the indicate time, the resulting suspension was added dropwise over 60 s to a solution of (1E,2E)-N_{1,}N₂-bis(2,4,4-trimethylpentan-2-yl)ethane-1,2-diimine (2.80 g, 10.0 mmol) in toluene (10.0 mL) in a 100 mL round-bottom flask at room temperature with continuous stirring. The resulting reaction mixture was stirred at 60° C. for 20 h. After the indicated time, diethyl ether (50 mL) was added and the reaction mixture was stirred for 30 min. The resulting precipitate was filtered, washed with a minimum of diethyl ether and dried under vacuum to obtain the title product as white solid in (2.41 g, 73% yield). Mp=246-247° C. ¹H NMR (500 MHz, CDCl₃) δ10.63 (s, 1H), 7.58 (d, J=0.7 Hz, 2H), 2.04 (s, 4H), 1.78 (s, 12H), 0.78 (s, 18H). ¹³C NMR (126 MHz, CDCl₃) δ136.68, 119.95, 64.19, 54.44, 31.95, 30.97, 30.72. IR (KBr, v, cm⁻¹): 3029, 2953, 2905, 2868, 1737, 1538, 1469, 1380, 1217, 1144, 905, 821, 674. HRMS (ESI) m/z: [M−Cl]⁺ Calcd for C₁₉H₃₇N₂ 293.2951; Found 293.2961.

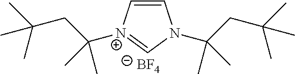

1,3-bis(2,4,4-trimethylpentan-2-yl)-1H-imidazol-3-ium tetrafluoroborate (1-BF₄). A 100 mL round-bottomed flask equipped with a stir bar was charged with (1E,2E)-N_{1,}N₂-bis(2,4,4-trimethylpentan-2-yl)ethane-1,2-diimine (1.4 g, 5.0 mmol), paraformaldehyde (152.0 mg, 5.0 mmol) and toluene (10.0 mL) at room temperature and the resulting mixture was stirred at 40° C. for 1 h. After the indicated time, the reaction mixture was cooled down to room temperature. HBF₄ (aq., 48% w/w, 0.65 mL, 5.0 mmol) was added with continuous stirring over 60 s, and the resulting mixture was stirred at 40° C. for 15 h. After the indicated time, the reaction mixture was cooled down to room temperature and concentrated. The resulting precipitate was filtered, washed with a minimum of diethyl ether and dried under vacuum to obtain the title product as white solid in (1.55 g, 82% yield). Mp=159-160° C. ¹H NMR (500 MHz, CDCl₃) δ9.00 (s, 1H), 7.54 (d, J=1.5 Hz, 2H), 1.96 (s, 4H), 1.75 (s, 12H), 0.85 (s, 18H). ¹³C NMR (126 MHz, CDCl₃) δ133.50, 120.61, 64.32, 54.45, 32.04, 30.99, 30.08. ¹⁹F NMR (471 MHz, CDCl₃) δ−150.64. IR (KBr, v, cm⁻¹): 3152, 2952, 1546, 1476, 1219, 1052, 1036, 669, 521. HRMS (ESI) m/z: [2M−BF₄]⁺ Calcd for C₃₈H₇₄N₄BF₄ 673.5944; Found 673.5970.

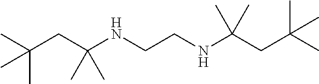 [SMILES: CC(C)(C)CC(C)(C)NCCNC(C)(C)CC(C)(C)C]

N¹,N²-bis(2,4,4-trimethylpentan-2-yl)ethane-1,2-diamine (3-1). A 250 mL round-bottomed flask equipped with a stir bar was charged with (1E,2E)-N_{1,}N₂-bis(2,4,4-trimethylpentan-2-yl)ethane-1,2-diimine (5.61 g, 20.0 mmol), methanol (20.0 mL), and THF (40.0 mL). NaBH₄ (6.05 g, 160.0 mmol) was added slowly at room temperature with vigorous stirring, and the resulting reaction mixture was stirred for 3 h at room temperature. After the indicated time, the reaction mixture was diluted with diethyl ether (50 mL), washed with saturated NH₄Cl (aq., 50 mL), and the aqueous layer was extracted with diethyl ether (3×20 mL). The combined organic layers were dried under vacuum to afford the title product as a white solid (5.16 g, 91% yield). Mp=30-31° C. ¹H NMR (500 MHz, CDCl₃) δ2.59 (s, 4H), 1.39 (s, 4H), 1.09 (s, 12H), 0.97 (s, 17H). ¹³C NMR (126 MHz, CDCl₃) δ54.27, 53.88, 42.98, 32.12, 31.96, 29.17. IR (KBr, v, cm⁻¹): 3295, 2953, 2906, 1472. HRMS (ESI) m/z: [M+H]⁺ Calcd for C₁₈H₄₁N₂ 285.3264; Found 285.3265.

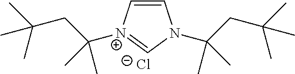 [SMILES: CC(C)(C)CC(C)(C)n1cc[n+](C(C)(C)CC(C)(C)C)c1 ; [Cl-]]

1,3-bis(2,4,4-trimethylpentan-2-yl)-4,5-dihydro-1H-imidazol-3-ium chloride (2-Cl). A mixture of N¹,N²-bis(2,4,4-trimethylpentan-2-yl)ethane-1,2-diamine (4.27 g, 15.0 mmol) and HCl (aq., 1.0 N, 50.0 mL) was stirred at room temperature for 3 h. After the indicated time, the resulting precipitate was filtered, washed with a minimum of diethyl ether and dried under vacuum to afford a diammonium salt as a white solid (5.14 g, 96% yield). Next, a 100 mL round-bottomed flask equipped with a stir bar was charged with triethyl orthoformate (8.3 mL, 50.0 mmol), the diammonium salt (1.79 g, 5.0 mmol) and formic acid (2 drops) at room temperature. The resulting reaction mixture was stirred at 125° C. for 36 h. After the indicated time, the reaction was cooled to room temperature and concentrated to form a white precipitate. The resulting precipitate was filtered, washed with a minimum of diethyl ether and dried under vacuum to obtain the title product as white solid (1.28 g, 77% yield). Mp=210-211° C. ¹H NMR (500 MHz, CDCl₃) δ8.81 (s, 1H), 4.00 (s, 4H), 1.63 (s, 4H), 1.54 (s, 12H), 0.91 (s, 18H). ¹³C NMR (126 MHz, CDCl₃) δ155.15, 61.08, 51.27, 45.76, 31.80, 31.40, 28.82. IR (KBr, v, cm⁻¹): 3451, 3389, 2947, 2905, 2870, 1622, 1464, 1292, 1211, 1135, 503. HRMS (ESI) m/z: [M−Cl]⁺ Calcd for C₁₉H₃₉N₂ 295.3108; Found 295.3117.

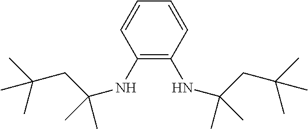 [SMILES: CC(C)(C)CC(C)(C)Nc1ccccc1NC(C)(C)CC(C)(C)C]

N¹,N²-bis(2,4,4-trimethylpentan-2-yl)benzene-1,2-diamine. An oven dried Schlenk tube (100 mL) equipped with a stir bar was charged with Pd₂(dba)₃ (183 mg, 0.20 mmol, 4.0 mol %), rac-BINAP (249 mg, 0.40 mmol, 8.0 mol %), toluene (12.0 mL), and heated at 135° C. for 15 min. The reaction mixture was then cooled to room temperature, tert-octylamine (3.23 g, 25.0 mmol), o-dibromobenzene (1.18 g, 5.0 mmol), NaOt-Bu (1.92 g, 20.0 mmol) and toluene (24.0 mL) were added, and heated at 135° C. for 60 h. After the indicated time, the reaction mixture was cooled down at room temperature, diluted with EtOAc (100 mL) and washed with aqueous NaOH (2.0 N, 1×100 mL). The organic layer was dried, filtered and concentrated. The pure product was after purification by chromatography on silica gel (EtOAc/hexane=1/15) as a brown oil (1.45 g, 87% yield). ¹H NMR (500 MHz, CDCl₃) δ6.90-6.86 (m, 2H), 6.78-6.68 (m, 2H), 3.74 (s, 2H), 1.69 (s, 4H), 1.32 (s, 12H), 1.06 (s, 18H). ¹³C NMR (126 MHz, CDCl₃) δ138.71, 120.56, 119.79, 56.04, 54.86, 32.06, 29.97. IR (KBr, v, cm⁻¹): 2950, 2901, 2868, 1596, 1509, 1473, 1218, 740, 488. HRMS (ESI) m/z: [M+H]⁺ Calcd for C₂₂H₄₁N₂ 333.3264; Found 333.3269.

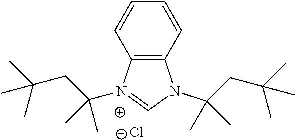 [SMILES: CC(C)(C)CC(C)(C)n1c[n+](C(C)(C)CC(C)(C)C)c2ccccc21 ; [Cl-]]

1,3-bis(2,4,4-trimethylpentan-2-yl)-1H-benzoidlimidazol-3-ium chloride [ItOct-bimy·HCl]. An oven dried Schlenk tube (100 mL) equipped with a stir bar was charged with diamine (1.66 g, 5.0 mmol), triethyl orthoformate (15.0 mL) and concentrated HCl (12 N, 0.5 mL, 6.0 mmol), and the resulting reaction mixture was stirred at 80° C. for 60 h. After the indicated time, the reaction mixture was concentrated to half volume, hot toluene (5.0 mL) and diethyl ether (5.0 mL) were added, and allowed to precipitate at room temperature. The crystalline precipitate appeared after 30 min, at which point the precipitation was increased by scratching the flask with spatula and adding diethyl ether (10 mL). The formed precipitate was filtered and dried to obtain the pure product as a white solid (1.05 g, 55% yield). Mp=169-170° C. ¹H NMR (500 MHz, CDCl₃) δ9.73 (s, 1H), 7.93 (dd, J=6.4, 3.2 Hz, 2H), 7.55 (dd, J=6.4, 3.1 Hz, 2H), 2.21 (s, 4H), 2.13 (s, 12H), 0.85 (s, 18H). ¹³C NMR (126 MHz, CDCl₃) δ140.93, 132.09, 126.02, 117.05, 66.61, 51.72, 32.07, 31.17, 29.96. IR (KBr, v, cm⁻¹): 3114, 2945, 1541, 1470, 1393, 1315, 1276, 1219, 748. HRMS (ESI) m/z: [M−Cl]⁺ Calcd for C₂₃H₃₉N₂ 343.3108; Found 343.3110.

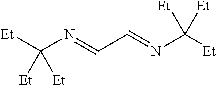 [SMILES: CCC(CC)(CC)/N=C/C=N/C(CC)(CC)CC]

N¹,N²-bis(3-ethylpentan-3-yl)ethane-1,2-diimine. A round-bottomed flask (250 mL) equipped with a stir bar was charged with 3-ethylpentan-3-amine (2.30 g, 20.0 mmol), glyoxal (aq., 40% w/w, 2.5 mL, 21.8 mmol) and hexane (50 mL). The resulting mixture was stirred at room temperature for 5 h. After the indicated time, the reaction mixture was dried, filtered and concentrated to obtain the pure product as colorless oil (2.25 g, 89% yield). ¹H NMR (500 MHz, CDCl₃) δ7.85 (s, 2H), 1.58 (q, J=7.5 Hz, 12H), 0.75 (t, J=7.5 Hz, 18H). ¹³C NMR (126 MHz, CDCl₃) δ159.57, 65.50, 28.56, 7.99. IR (KBr, v, cm⁻¹): 2970, 2931, 2880, 1713, 1629, 1381, 1163, 1117, 975, 934, 894. HRMS (ESI) m/z: [M+H]⁺ Calcd for C₁₆H₃₃N₂ 253.2638; Found 253.2642.

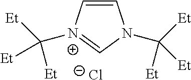 [SMILES: CCC(CC)(CC)n1cc[n+](C(CC)(CC)CC)c1 ; [Cl-]]

1,3-bis(3-ethylpentan-3-yl)-1H-imidazol-3-ium chloride [ItHept·HCl]. An oven dried round-bottomed flask (100 mL) equipped with a stir bar was charged with diimine (2.0 g, 7.92 mmol), paraformaldehyde (261 mg, 8.71 mmol) and EtOAc (10 mL), and stirred for 30 min at room temperature. TMSC₁ (0.93 mL, 7.92 mmol) was then added dropwise over 60 s, and the reaction mixture was stirred at 40° C. for 15 h. After the indicated time, the reaction mixture was cooled down to below 10° C., the formed precipitate was filtered and dried to obtain pure product as a white solid (1.28 g, 54% yield). Mp=219-220° C. ¹H NMR (500 MHz, CDCl₃) δ10.46 (s, 1H), 7.46 (d, J=1.5 Hz, 2H), 2.12 (q, J=7.4 Hz, 12H), 0.74 (t, J=7.4 Hz, 18H). ¹³C NMR (126 MHz, CDCl₃) δ136.27, 120.37, 69.98, 28.68, 7.45. IR (KBr, v, cm⁻¹): 3526, 3043, 2966, 2879, 1528, 1458, 1162, 1122, 893, 853, 813, 670. HRMS (ESI) m/z: [M−Cl]⁺ Calcd for C₁₇H₃₃N₂ 265.2638; Found 265.2642.

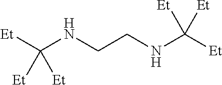 [SMILES: CCC(CC)(CC)NCCNC(CC)(CC)CC]

N¹,N²-bis(3-ethylpentan-3-yl)ethane-1,2-diamine. An oven dried round-bottomed flask (250 mL) equipped with a stir bar was charged with diimine (2.0 g, 7.92 mmol), methanol (10 mL) and THF (20 mL). NaBH₄ (2.4 g, 63.4 mmol) was added slowly at room temperature with vigorous stirring, and the resulting reaction mixture was allowed to stir at room temperature for 5 h. After the indicated time, the reaction mixture was diluted with diethyl ether (50 mL), washed with saturated NH₄Cl (aq., 50 mL), the aqueous layer was extracted with diethyl ether (3×20 mL), and the combined organic layers were dried, filtered and concentrated to afford pure product as colorless oil (1.65 g, 81% yield). ¹H NMR (500 MHz, CDCl₃) δ2.47 (s, 4H), 1.29 (q, J=7.5 Hz, 12H), 0.76 (t, J=7.5 Hz, 18H). ¹³C NMR (126 MHz, CDCl₃) δ56.49, 41.99, 27.20, 7.65. IR (KBr, v, cm⁻¹): 2961, 2924, 2876, 1459, 1175, 913, 738, 676. HRMS (ESI) m/z: [M+H]⁺ Calcd for C₁₆H₃₇N₂ 257.2951; Found 257.2955.

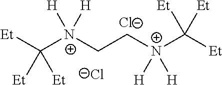 [SMILES: CCC(CC)(CC)[NH2+]CC[NH2+]C(CC)(CC)CC ; [Cl-] ; [Cl-]]

N¹,N²-bis(3-ethylpentan-3-yl)ethane-1,2-diamine-di-hydrochloride. A mixture of diamine (1.5 g, 5.85 mmol) and aqueous HCl (2.0 N, 15 mL) was stirred at room temperature for 5 h. After the indicate time, the reaction mixture was concentrated to half volume and cooled down to below 10° C. The formed precipitate was filtered and dried to afford pure product as a white solid (1.58 g, 82% yield). Mp=281-282° C. ¹H NMR (500 MHz, DMSO) δ8.92 (s, 4H), 3.32 (s, 4H), 1.63 (q, J=7.3 Hz, 12H), 0.90 (t, J=7.4 Hz, 18H). ¹³C NMR (126 MHz, DMSO) δ65.42, 37.23, 25.33, 7.40. IR (KBr, v, cm⁻¹): 3327, 2971, 2946, 2882, 2742, 2568, 2454, 1738, 1606, 1459, 1017, 901. HRMS (ESI) m/z: [2M−H₂Cl₃]⁺ Calcd for C₃₂H₇₄N₄Cl 549.5597; Found 549.5587.

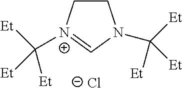 [SMILES: CCC(CC)(CC)N1C=[N+](C(CC)(CC)CC)CC1 ; [Cl-]]

1,3-bis(3-ethylpentan-3-yl)-4,5-dihydro-1H-imidazolium chloride [SItHept·HCl]. An oven dried Schlenk tube (100 mL) equipped with a stir bar was charged with diamine·HCl salt (1.5 g, 4.55 mmol), triethyl orthoformate (10 mL, 75.7 mmol) and formic acid (5 drops), and stirred at 130° C. for 60 h. Note that during this time, the mixture turned clear after 12 h and turned brown after 36 h. After the indicate time, the reaction mixture was concentrated to half volume, (note that during this time white precipitate was formed), and diethyl ether (20 mL) was added. The formed precipitate was filtered and dried to obtain pure product as a while solid. Yield 86% (1.18 g, 3.89 mmol). (1.32 mmol scale: 87% yield). Mp=162-163° C. ¹H NMR (500 MHz, CDCl₃) δ8.39 (s, 1H), 4.04 (s, 4H), 1.74 (q, J=7.4 Hz, 12H), 0.81 (t, J=7.4 Hz, 18H). ¹³C NMR (126 MHz, CDCl₃) δ155.52, 66.18, 45.86, 26.60, 7.67. IR (KBr, v, cm⁻¹): 3362, 2968, 2937, 2882, 1607, 1463, 1295, 1276, 1157, 1027, 1014, 479. HRMS (ESI) m/z: [M−Cl]⁺ Calcd for C₁₇H₃₅N₂ 267.2795; Found 267.2798.

### Example 2: Synthesis of NHC Catalyst Complexes

With facile access to ItOct (1) in hand, focused was placed on comprehensive evaluation of steric and electronic properties of this novel NHC ligand (Scheme 4). As shown, the gold complex [Au(ItOct)Cl] was prepared using LiHMDS/THF, while the method using K₂CO₃/acetone gave slightly lower yields. Moreover, [Ag(ItOct)Cl] and [Cu(ItOct)Cl] were prepared using Ag₂O/CuCl and K₂CO₃ in 1,4-dioxane at 80° C. The selenium adduct [Se(ItOct)] was synthesized under the same conditions using selenium/K₂CO₃ at 80° C., while the Pd(II) complexes [Pd(ItOct)(allyl)Cl] and [Pd(ItOct)(3-Cl-py)Cl₂] were prepared from the palladium allyl dimer [{Pd(allyl)(μ-Cl)}₂] and PdCl₂/3-Cl-py in the presence of LiHMDS and K₂CO₃, respectively. It should be noted that all NHC ligands and products NHC catalyst complexes were found to be stable to air and moisture.

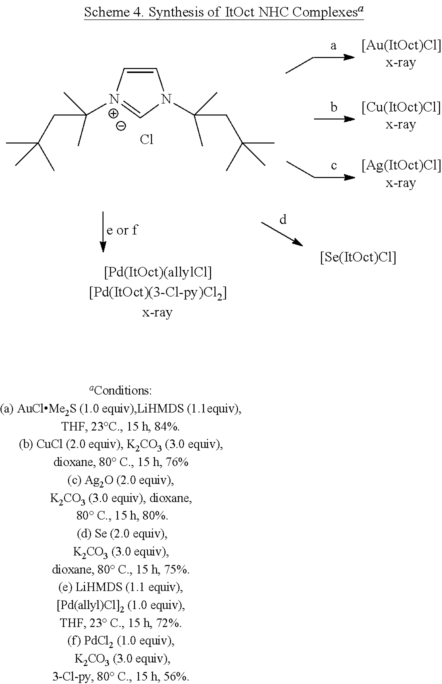

Representative complexes using the imidazolinium precursor SItOct (2) were also prepared (Scheme 5). As shown, the synthesis of [Au(SItOct)Cl], [Se(SItOct)], and [Pd(ItOct)(allyl)Cl] proceeded uneventfully under the same conditions as developed from ItOct·HCl (Scheme 2A). The δ_{Se} value of 298.2 ppm and the ¹J_{CH} value of 200.80 Hz are consistent with an increased π-acceptance and σ-donation of the saturated SItBu, as expected.

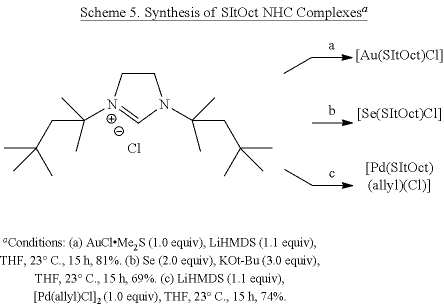

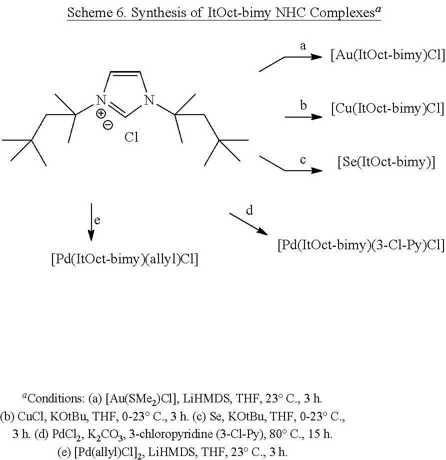

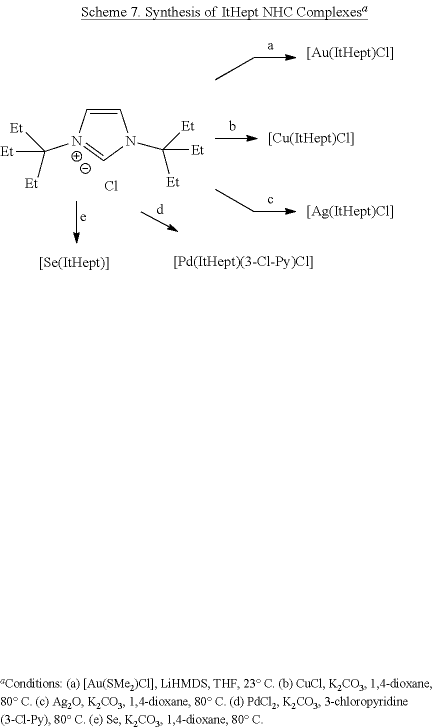 [SMILES: CCC(CC)(CC)n1cc[n+](C(CC)(CC)CC)c1 ; Cl]

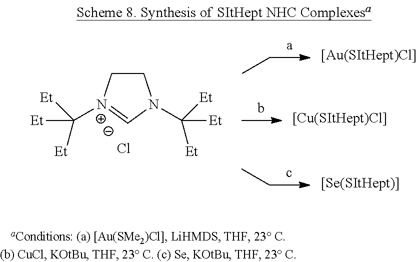

### Synthesis of NHC Catalyst Complexes

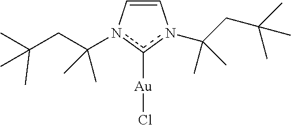

1,3-bis(2,4,4-trimethylpentan-2-yl)-2,3-dihydro-1H-imidazol-2-ylidene gold(I) chloride [Au(ItOct)Cl]. An oven-dried flask equipped with a stir bar was charged with ItOct·HCl (1-Cl) (39.5 mg, 0.12 mmol, 1.2 equiv), placed under a positive pressure of argon and subjected to three evacuation/backfilling cycles under high vacuum. THF (0.5 mL) and LiHMDS (1.0 M in THF, 0.11 mL, 0.11 mmol, 1.1 equiv) were added and the reaction mixture was stirred at room temperature for 2 h. After the indicated time, a solution of AuCl·SMe2 (29.5 mg, 0.1 mmol, 1.0 equiv) in THF (0.5 mL) was added dropwise, and the resulting mixture was stirred at room temperature for 15 h. After the indicated time, the reaction mixture was diluted with CH₂Cl₂ (10 mL) and filtered. The solution was collected and concentrated. The title product was obtained by trituration from hexanes as a white solid (43.9 mg, 84% yield). Mp=219-220° C. ¹H NMR (500 MHz, CDCl₃) δ7.10 (s, 2H), 2.64 (s, 4H), 1.85 (s, 12H), 0.82 (s, 18H). ¹³C NMR (126 MHz, CDCl₃) δ170.34, 117.39, 62.57, 54.95, 32.36, 32.28, 31.49. IR (KBr, v, cm⁻¹): 3166, 2948, 2868, 1739, 1568, 1472, 1407, 1390, 1363, 1215, 1176, 730, 701, 636. HRMS (ESI) m/z: [2M+K]⁺ Calcd for C₃₈H₇₂N₄Cl₂Au₂K 1087.4097; Found 1087.4168.

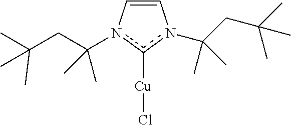

1,3-bis(2,4,4-trimethylpentan-2-yl)-2,3-dihydro-1H-imidazol-2-ylidene copper(I) chloride [Cu(ItOct)Cl]. An oven-dried flask equipped with a stir bar was charged with ItOct·HCl (1-Cl) (32.9 mg, 0.1 mmol, 1.0 equiv), CuCl (19.8 mg, 0.2 mmol, 2.0 equiv) and K₂CO₃ (41.4 mg, 0.3 mmol, 3.0 equiv), placed under a positive pressure of argon, and subjected to three evacuation/backfilling cycles under high vacuum. 1,4-dioxane (1.0 mL) was added, and the reaction mixture was stirred at 80° C. for 15 h. After the indicated time, the reaction mixture was diluted with CH₂Cl₂ (10 mL) and filtered. The solution was collected and concentrated. The title product was obtained by trituration from hexanes as a white solid (29.8 mg, 76% yield). Mp=194-195° C. ¹H NMR (500 MHz, CDCl₃) δ7.04 (s, 2H), 2.33 (s, 4H), 1.80 (s, 12H), 0.82 (s, 18H). ¹³C NMR (126 MHz, CDCl₃) δ175.12, 117.40, 61.34, 56.52, 32.41, 32.29, 31.43. IR (KBr, v, cm⁻¹): 3159, 2949, 2869, 1470, 1400, 1217, 1174, 730, 698, 638. HRMS (ESI) m/z: [2M−Cl]⁺ Calcd for C₃₈H₇₂N₄ClCu₂ 747.4013; Found 747.4021.

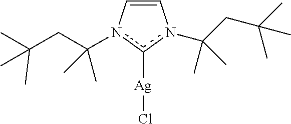

1,3-bis(2,4,4-trimethylpentan-2-yl)-2,3-dihydro-1H-imidazol-2-ylidene silver(I) chloride [Ag(ItOct)Cl]. An oven-dried flask equipped with a stir bar was charged with ItOct·HCl (1-Cl) (32.9 mg, 0.1 mmol, 1.0 equiv), Ag₂O (46.3 mg, 0.2 mmol, 2.0 equiv) and K₂CO₃ (41.4 mg, 0.3 mmol, 3.0 equiv), placed under a positive pressure of argon, and subjected to three evacuation/backfilling cycles under high vacuum. 1,4-dioxane (1.0 mL) was added, and the reaction mixture was stirred at 80° C. for 15 h. After the indicated time, the reaction mixture was diluted with CH₂Cl₂ (10 mL) and filtered. The solution was collected and concentrated. The title product was obtained by trituration from hexanes as a white solid (35.0 mg, 80% yield). Mp=169-170° C. ¹H NMR (500 MHz, CDCl₃) δ7.11 (d, J=1.7 Hz, 2H), 2.26 (s, 4H), 1.76 (s, 12H), 0.79 (s, 18H). ¹³C NMR (126 MHz, CDCl₃) δ178.03 (dd, J^{Ag}=257.1, 18.5 Hz), 117.85 (d, J^{Ag}=8.0 Hz), 61.43, 56.34, 32.33, 32.27, 31.46. IR (KBr, v, cm⁻¹): 3152, 2949, 1614, 1471, 1391, 1366, 1216, 1173, 1134, 732, 691, 635. HRMS (ESI) m/z: [2M−Cl]⁺ Calcd for C₃₈H₇₂N₄ClAg₂ 835.3535; Found 835.3559.

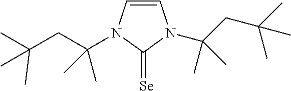 [SMILES: CC(C)(C)CC(C)(C)n1ccn(C(C)(C)CC(C)(C)C)c1=[Se]]

1,3-bis(2,4,4-trimethylpentan-2-yl)-2,3-dihydro-1H-imidazol-2-ylidene selenide [Se(ItOct)]. An oven-dried flask equipped with a stir bar was charged with ItOct·HCl (1-Cl) (32.9 mg, 0.1 mmol, 1.0 equiv), selenium (15.8 mg, 0.2 mmol, 2.0 equiv), and K₂CO₃ (41.4 mg, 0.3 mmol, 3.0 equiv), placed under a positive pressure of argon, and subjected to three evacuation/backfilling cycles under high vacuum. 1,4-dioxane (1.0 mL) was added, and the reaction mixture was stirred at 80° C. for 15 h. After the indicated time, the reaction mixture was diluted with CH₂Cl₂ (10 mL) and filtered. The solution was collected and concentrated. The title product was obtained by trituration from hexanes as a white solid (28.0 mg, 75% yield). Mp=131-132° C. ¹H NMR (500 MHz, CDCl₃) δ7.01 (s, 2H), 2.77 (s, 4H), 1.85 (s, 12H), 0.86 (s, 18H). ¹³C NMR (126 MHz, CDCl₃) δ151.95, 116.68, 64.65, 47.44, 32.07, 31.48, 30.87. ⁷⁷Se NMR (95 MHz, CDCl₃) δ216.72. IR (KBr, v, cm⁻¹): 2948, 2900, 2867, 1740, 1476, 1365, 1341, 1209, 1176, 1131, 1043. HRMS (ESI) m/z: [M+Na]⁺ Calcd for C₁₉H₃₆N₂SeNa 395.1937; Found 395.1949.

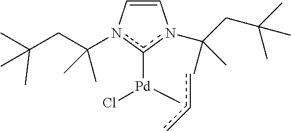

1,3-bis(2,4,4-trimethylpentan-2-yl)-2,3-dihydro-1H-imidazol-2-ylidene palladium(II) (allyl) chloride [Pd(ItOct)(allyl)Cl]. An oven-dried flask equipped with a stir bar was charged with ItOct·HCl (39.5 mg, 0.12 mmol, 1.2 equiv), placed under a positive pressure of argon and subjected to three evacuation/backfilling cycles under high vacuum. THF (0.5 mL) and LiHMDS (1.0 M in THF, 0.11 mL, 0.11 mmol, 1.1 equiv) were added and the resulting reaction mixture was stirred at room temperature for 2 h. After the indicated time, [{Pd(allyl)Cl}₂] (29.5 mg, 0.1 mmol, 1.0 equiv) in THF (0.5 mL) was added and the reaction mixture was stirred at room temperature for 15 h. After the indicated time, the reaction mixture was diluted with CH₂Cl₂ (10 mL) and filtered. The solution was collected and concentrated. The title product was obtained by trituration from hexanes as a yellow solid (34.1 mg, 72% yield). Mp=152-153° C. ¹H NMR (500 MHz, CDCl₃) δ7.11 (dd, J=9.5, 2.1 Hz, 2H), 5.26-5.17 (m, 1H), 4.10 (dd, J=7.3, 2.2 Hz, 1H), 3.39 (d, J=6.5 Hz, 1H), 3.25 (d, J=13.2 Hz, 1H), 2.26 (d, J=11.7 Hz, 1H), 2.13-2.05 (m, 4H), 1.99 (s, 2H), 1.92-1.85 (m, 7H), 1.69 (s, 3H), 0.83 (d, J=10.8 Hz, 18H). ¹³C NMR (126 MHz, CDCl₃) δ177.52, 119.36, 119.03, 112.42, 70.06, 62.63, 62.58, 55.72, 55.16, 52.46, 32.68, 32.62, 32.50, 32.12, 32.08, 32.06, 31.56, 31.47. IR (KBr, v, cm⁻¹): 2950, 1739, 1459, 1367, 1210, 1169. HRMS (ESI) m/z: [2M−Cl]⁺ Calcd for C₄₄H₈₂N₄ClPd₂ 915.4303; Found 915.4298.

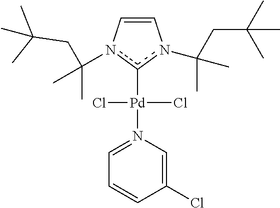

3-chloropyridine [1,3-bis(2,4,4-trimethylpentan-2-yl)-2,3-dihydro-1H-imidazol-2-ylidene] palladium(II) dichloride [Pd(ItOct)(3-Cl-py)Cl_{2].} An oven-dried flask equipped with a stir bar was charged with ItOct·HCl (32.9 mg, 0.1 mmol, 1.0 equiv), PdCl₂ (17.7 mg, 0.1 mmol, 1.0 equiv) and K₂CO₃ (41.4 mg, 0.3 mmol, 3.0 equiv), placed under a positive pressure of argon and subjected to three evacuation/backfilling cycles under high vacuum. 3-chloropyridine (0.5 mL) was added, and the reaction mixture was stirred at 80° C. for 15 h. After the indicated time, the reaction mixture was diluted with CH₂Cl₂ (10 mL) and filtered. The solution was collected and concentrated. The title product was obtained by trituration from hexanes as a yellow solid (37.9 mg. 56% yield). Mp=199-200° C. ¹H NMR (500 MHz, CDCl₃) δ9.01 (d, J=2.2 Hz, 1H), 8.94 (dd, J=5.4, 1.0 Hz, 1H), 7.74 (ddd, J=8.2, 2.1, 1.3 Hz, 1H), 7.30 (dd, J=8.2, 5.5 Hz, 1H), 7.22 (s, 2H), 2.46 (s, 12H), 2.11 (s, 4H), 0.97 (s, 18H). ¹³C NMR (126 MHz, CDCl₃) δ151.04, 150.09, 141.33, 138.12, 132.94, 125.19, 121.04, 63.89, 55.88, 33.22, 32.21, 31.62. IR (KBr, v, cm⁻¹): 3136, 2957, 1592, 1461, 1368, 1116, 1048, 797, 744, 688, 647. HRMS (ESI) m/z: [2M−2(Cl+NC₅H₄Cl)]⁺ Calcd for C₃₄H₇₂N₄Cl₂Pd₂ 868.3194; Found 868.3179.

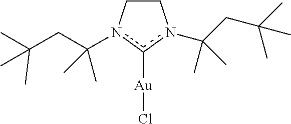

1,3-bis(2,4,4-trimethylpentan-2-yl)imidazolidin-2-ylidene gold(I) chloride [Au(SItOct)Cl]. An oven-dried flask equipped with a stir bar was charged with SItOct·HCl (39.7 mg, 0.12 mmol, 1.2 equiv), placed under a positive pressure of argon and subjected to three evacuation/backfilling cycles under high vacuum. THF (0.5 mL) and LiHMDS (1.0 M in THF, 0.11 mL, 0.11 mmol, 1.1 equiv) were added and the reaction mixture was stirred at room temperature for 2 h. After the indicated time, a solution of AuCl·SMe2 (29.5 mg, 0.1 mmol, 1.0 equiv) in THF (0.5 mL) was added dropwise, and the resulting mixture was stirred at room temperature for 15 h. After the indicated time, the reaction mixture was diluted with CH₂Cl₂ (10 mL) and filtered. The solution was collected and concentrated. The title product was obtained by trituration from hexanes as a white solid (42.8 mg, 81% yield). ¹H NMR (500 MHz, CDCl₃) δ3.64 (s, 4H), 2.48 (s, 4H), 1.62 (s, 12H), 1.01 (s, 18H). ¹³C NMR (126 MHz, CDCl₃) δ193.23, 60.39, 55.22, 46.69, 32.33, 32.14, 31.47. Mp=154-155° C. IR (KBr, v, cm⁻¹): 2948, 2901, 1470, 1436, 1422, 1267, 1212, 1128, 613, 600. HRMS (ESI) m/z: [M+(MeCN)−Cl]⁺ Calcd for C₂₁H₄₁N₃Au 532.2961; Found 532.2980.

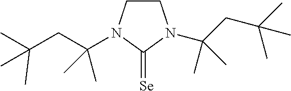 [SMILES: CC(C)(C)CC(C)(C)N1CCN(C(C)(C)CC(C)(C)C)C1=[Se]]

1,3-bis(2,4,4-trimethylpentan-2-yl)imidazolidin-2-ylidene selenide [(SItOct)Se]. An oven-dried flask equipped with a stir bar was charged with SItOct·HCl (33.1 mg, 0.1 mmol, 1.0 equiv), selenium (15.8 mg, 0.2 mmol, 2.0 equiv), and KO^{t}Bu (33.7 mg, 0.3 mmol, 3.0 equiv), placed under a positive pressure of argon, and subjected to three evacuation/backfilling cycles under high vacuum. THF (1.0 mL) was added, and the reaction mixture was stirred at room temperature for 15 h. After the indicated time, the reaction mixture was diluted with CH₂Cl₂ (10 mL) and filtered. The solution was collected and concentrated. The title product was obtained by trituration from hexanes as a white solid (25.6 mg, 69% yield). ¹H NMR (500 MHz, CDCl₃) δ3.53 (s, 4H), 2.59 (s, 4H), 1.64 (s, 12H), 1.03 (s, 18H). ¹³C NMR (126 MHz, CDCl₃) δ178.62, 62.24, 48.60, 45.99, 32.00, 31.90, 30.38. ⁷⁷Se NMR (95 MHz, CDCl₃) δ298.15. Mp=113-114° C. IR (KBr, v, cm⁻¹): 2950, 2899, 2866, 1471, 1393, 1297, 1265, 1210, 1126. HRMS (ESI) m/z: [2M+Na]⁺ Calcd for C₃₈H₇₆N₄Se₂Na 771.4300; Found 771.4326.

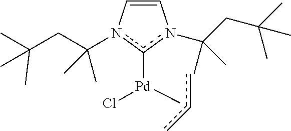

1,3-bis(2,4,4-trimethylpentan-2-yl)imidazolidin-2-ylidene palladium(II) (allyl) chloride [Pd(SItOct)(allyl)Cl]. An oven-dried flask equipped with a stir bar was charged with SItOct·HCl (39.7 mg, 0.12 mmol, 1.2 equiv), placed under a positive pressure of argon and subjected to three evacuation/backfilling cycles under high vacuum. THF (0.5 mL) and LiHMDS (1.0 M in THF, 0.11 mL, 0.11 mmol, 1.1 equiv) were added and the resulting reaction mixture was stirred at room temperature for 2 h. After the indicated time, [{Pd(allyl)Cl}₂] (29.5 mg, 0.1 mmol, 1.0 equiv) in THF (0.5 mL) was added and the reaction mixture was stirred at room temperature for 15 h. After the indicated time, the reaction mixture was diluted with CH₂Cl₂ (10 mL) and filtered. The solution was collected and concentrated. The title product was obtained by trituration from hexanes as a yellow solid (35.5 mg, 74% yield). Mp=145-146° C. ¹H NMR (500 MHz, CDCl₃) δ5.24-5.15 (m, 1H), 4.08 (dd, J=7.1, 1.1 Hz, 1H), 3.65-3.52 (m, 4H), 3.40 (d, J=6.3 Hz, 1H), 3.25 (d, J=13.3 Hz, 1H), 2.22 (d, J=11.6 Hz, 1H), 2.01 (s, 3H), 1.83-1.71 (m, 9H), 1.58 (s, 4H), 1.01 (d, J=13.7 Hz, 18H). ¹³C NMR (126 MHz, CDCl₃) δ206.92, 112.74, 69.82, 60.84, 60.68, 53.21, 52.88, 51.12, 46.16, 45.92, 31.97, 31.94, 31.91, 31.55, 31.10, 30.99. IR (KBr, v, cm⁻¹): 2949, 1736, 1421, 1362, 1274, 1209. HRMS (ESI) m/z: [2M−Cl]⁺ Calcd for C₄₄H₈₆N₄ClPd₂ 919.4616; Found 919.4609.

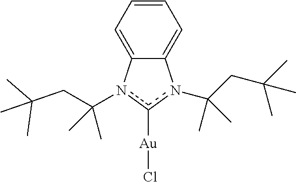

1,3-bis(2,4,4-trimethylpentan-2-yl)benzimidazolidin-2-ylidene gold(I) chloride [Au(ItOct·bimy)Cl]. An oven-dried flask equipped with a stir bar was charged with ItOct-bimy·HCl (45.5 mg, 0.12 mmol, 1.2 equiv), placed under a positive pressure of argon and subjected to three evacuation/backfilling cycles under high vacuum. THF (0.5 mL) and LiHMDS (1.0 M in THF, 0.11 mL, 0.11 mmol, 1.1 equiv) were added and the reaction mixture was stirred at room temperature for 2 h. After the indicated time, a solution of AuClSMe₂ (29.5 mg, 0.1 mmol, 1.0 equiv) in THF (0.5 mL) was added dropwise, and the resulting mixture was stirred at room temperature for 15 h. After the indicated time, the reaction mixture was diluted with CH₂Cl₂ (10 mL) and filtered. The solution was collected and concentrated. The title product was obtained by trituration from hexanes as a white solid (49.4 mg, 86% yield). Mp=174-1175° C. ¹H NMR (500 MHz, CDCl₃) δ8.01-7.91 (m, 2H), 7.33-7.26 (m, 2H), 2.58 (s, 4H), 2.31 (s, 12H), 0.83 (s, 18H). ¹³C NMR (126 MHz, CDCl₃) δ179.82, 134.37, 122.93, 116.37, 65.92, 54.01, 34.27, 32.29, 31.37. IR (KBr, v, cm⁻¹) 2951, 2903, 1467, 1356, 1334, 1200, 741. HRMS (ES1) m/z: [2M−Cl]⁺ Calcd for C₄₆H₇₆N₄ClAu₂ 1113.5084; Found 1113.5049.

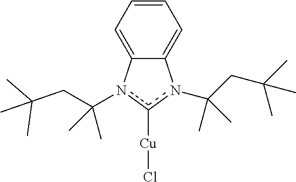

1,3-bis(2,4,4-trimethylpentan-2-yl)benzimidazolidin-2-ylidene copper(I) chloride [Cu(ItOct·bimy)Cl]. An oven-dried flask equipped with a stir bar was charged with ItOct-bimy·HCl (37.9 mg, 0.1 mmol, 1.0 equiv), CuCl (19.8 mg, 0.2 mmol, 2.0 equiv) and KO^{t}Bu (33.7 mg, 0.3 mmol, 3.0 equiv), placed under a positive pressure of argon, and subjected to three evacuation/backfilling cycles under high vacuum. THF (1.0 mL) was added, and the reaction mixture was stirred at room temperature for 3 h. After the indicated time, the reaction mixture was diluted with CH₂Cl₂ (10 mL) and filtered. The solution was collected and concentrated. The title product was obtained by trituration from hexanes as a white solid (27.4 mg, 62% yield), Mp=150-151° C. ¹H NMR (500 MHz, CDCl₃) δ7.86-7.79 (m, 2H), 7.32-7.26 (m, 2H), 2.26 (s, 4H), 2.20 (s, 12H), 0.80 (s, 18H). ¹³C NMR (126 MHz, CDCl₃) δ182.69, 134.58, 122.48, 116.09, 63.27, 52.27, 33.16, 32.17, 31.23. IR (KBr, v, cm⁻¹) 2946, 2903, 2866, 1469, 1319, 1202741. HRMS (ESI) m/z: [2M−Cl]⁺ Calcd for C₄₆H₇₆N₄ClCu₂ 847.4328; Found 847.4360.

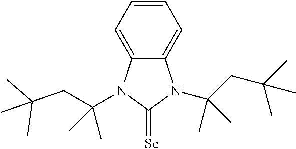 [SMILES: CC(C)(C)CC(C)(C)n1c(=[Se])n(C(C)(C)CC(C)(C)C)c2ccccc21]

1,3-bis(2,4,4-trimethylpentan-2-yl)₄,3-dihydro-2H-benzo[d]imidazole-2-selenone [Se(ItOct·bimy)]. An oven-dried flask equipped with a stir bar was charged with ItOct-bimy·HCl (37.9 mg, 0.1 mmol, 1.0 equiv), selenium (15.8 mg, 0.2 mmol, 2.0 equiv), and KO^{t}Bu (33.7 mg, 0.3 mmol, 3.0 equiv), placed under a positive pressure of argon, and subjected to three evacuation/backfilling cycles under high vacuum. THF (1.0 mL) was added, and the reaction mixture was stirred at room temperature for 3 h. After the indicated time, the reaction mixture was diluted with CH₂Cl₂ (10 mL) and filtered. The solution was collected and concentrated. The title product was obtained by trituration from hexanes as a white solid (21.9 mg, 52% yield). Mp=decomposed<100° C. ¹H NMR (500 CDCl₃) δ7.97-7.92 (m, 2H), 7.61-7.52 (m, 2H), 2.20 (s, 4H), 2.11 (s, 12H), 0.84 (s, 18H). ¹³C NMR (126 MHz, CDCl₃) δ131.88, 125.88, 116.88, 66.39, 51.55, 31.88, 30.95, 29.79. ⁷⁷Se NMR (95 MHz, CDCl₃) δ295.37. IR (KBr, v, cm⁻¹) 2950, 2868, 1675, 1600, 1469, 1306, 1212. HRMS (ESI) m/z: [M−Se]⁺ Calcd for C₂₃H₃₈N₂ 343.3108; Found 343.3125.

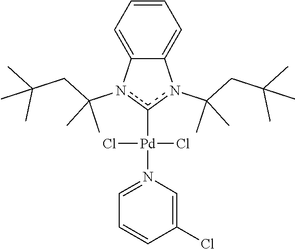

3-chloropyridine [1,3-bis(2,4,4-trimethylpentan-2-yl)-benzimidazol-2-ylidene] palladium(II) dichloride [Pd(ItOct·bimy)(3-Cl-Py)Cl₂]. An oven-dried flask equipped with a stir bar was charged with ItOct-bimy·HCl (37.9 mg, 0.1 mmol, 1.0 equiv), PdCl₂ (17.7 mg, 0.1 mmol, 1.0 equiv) and K₂CO₃ (41.4 mg, 0.3 mmol, 3.0 equiv), placed under a positive pressure of argon and subjected to three evacuation/backfilling cycles under high vacuum. 3-Chloropyridine (0.5 mL) was added, and the reaction mixture was stirred at 80° C. for 15 h. After the indicated time, the reaction mixture was diluted with CH₂Cl₂ (10 mL) and filtered. The solution was collected and concentrated. The title product was obtained by trituration from hexanes as a yellow solid (49.4 mg, 78% yield). Mp=92-93° C. ¹H NMR (500 MHZ, CDCl₃) δ9.03 (d, J=2.2 Hz, 1H), 8.96 (d, J=5.4 Hz, 1H), 7.86 (dd, J=6.3, 3.3 Hz, 2H), 7.77 (d, J=8.2 Hz, 1H), 7.32 (dd, J=8.2, 5.5 Hz, 1H), 7.21 (dd, J=6.4, 3.1 Hz, 2H), 2.86 (s, 12H), 2.37 (s, 4H), 0.99 (s, 18H). ¹³C NMR (126 MHz, CDCl₃) δ155.88, 150.67, 149.73, 137.94, 135.46, 132.77, 125.01, 121.47, 115.81, 65.99, 52.34, 32.80, 32.21, 31.31. IR (KBr, v, cm⁻¹) 2950, 2868, 1693, 1477, 1346, 1202, 742. HRMS (ESI) m/z: [M−C₅H₄NCl₂+2MeCN]⁺ Calcd for C₂₈H₄₂N₃Cl2Pd 598.1787; Found 598.1779.

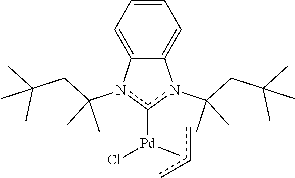

Allyl [1,3-bis(2,4,4-trimethylpentan-2-yl)benzimidazolidin-2-ylidene] palladium(II) chloride [Pd(ItOct·bimy)(allyl)Cl]. An oven-dried flask equipped with a stir bar was charged with ItOct-bimy·HCl (45.5 mg, 0.12 mmol, 1.2 equiv), placed under a positive pressure of argon and subjected to three evacuation/backfilling cycles under high vacuum. THF (0.5 mL) and LiHMDS (1.0 M in THF, 0.11 mL, 0.11 mmol, 1.1 equiv) were added and the resulting reaction mixture was stirred at room temperature for 2 h. After the indicated time, [{Pd(allyl)Cl}₂] (29.5 mg, 0.1 mmol, 1.0 equiv) in THF (0.5 mL) was added and the reaction mixture was stirred at room temperature for 3 h. After the indicated time, the reaction mixture was diluted with CH₂Cl₂ (10 mL) and filtered. The solution was collected and concentrated. The title product was obtained by trituration from hexanes as a yellow solid (28.9 mg, 55% yield). Mp=79-80° C. ¹H NMR (500 MHz, CDCl₃) δ7.90-7.69 (in, 2H), 7.20 (p, J=6.8 Hz, 2H), 5.45 (ddd, J=18.8, 12.4, 6.7 Hz, 1H), 4.16 (d, J=5.7 Hz, 1H), 4.11 (d, J=6.7 Hz, 2H), 3.35 (d,J=8.5 Hz, 1H), 3.03 (d, J=12.1 Hz, 2H), 2.40 (dd, J=31.9, 18.6 Hz, 6H), 2.28 (d, J=15.3 Hz, 1H), 2.23-2.16 (m, 6H), 2.10 (d,J=15.2 Hz, 1H), 0.89 (d,J=10.1 Hz, 18H). ¹³C NMR (126 MHz, CDCl₃) δ189.01, 135.69, 135.63, 121.21, 121.16, 115.74, 112.11, 111.21, 68.37, 64.21, 64.18, 62.98, 60.44, 52.20, 52.04, 51.12, 33.12, 32.57, 32.36, 32.18, 32.00, 31.99, 31.29, 21.10, 14.24. IR (KBr, v, cm⁻¹):2949, 2868, 1540, 1472, 1340, 1309, 1200, 744. HRMS (ESI) m/z: [2M−Cl]⁺ Calcd for C₅₂H₈₆N₄ClPd₂ 1015.4624; Found 1015.4595.

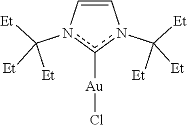

1,3-bis(3-ethylpentan-3-yl)-1H-1-imidazol-2-ylidene gold(I) chloride [Au(ItHept)Cl]. An oven-dried flask equipped with a stir bar was charged with ItHept·HCl (36.2 mg, 0.12 mmol, 1.2 equiv), placed under a positive pressure of argon and subjected to three evacuation/backfilling cycles under high vacuum. THF (0.5 mL) and. LiHMDS (1.0 M in THF, 0.11 mL, 0.11 mmol, 1.1 equiv) were added and the reaction mixture was stirred at room temperature for 2 h. After the indicated time, a solution of AuClSMe₂ (29.5 mg, 0.1 mmol, 1.0 equiv) in THF (0.5 mL) was added dropwise, and the resulting mixture was stirred at room temperature for 15 h. After the indicated time, the reaction mixture was diluted with CH₂Cl₂ (10 mL) and filtered. The solution was collected and concentrated. The title product was obtained by trituration from hexanes as a white solid (32.3 mg, 65% yield). Mp=130-131° C. ¹H NMR (500 MHz, CDCl₃) δ7.08 (s, 2H), 2.38 (q, J=7.2 Hz, 12H), 0.69 (t, J=7.3 Hz, 18H). ¹³C NMR (126 MHz, CDCl₃) δ168.94, 118.27, 68.07, 29.41, 7.55. IR (KBr, v, cm⁻¹) 3144, 2969, 1594, 1409, 1317, 1170, 1032, 994, 855, 743. HRMS (ESI) m/z: [2M−Cl]⁺ Calcd for C₃₄H₆₄N₄ClAu₂ 957.4145; Found 957.4144.

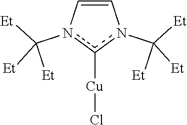

1,3-bis(3-ethylpentan-3-yl)-1H-imidazol-2-ylidene copper(I) chloride [Cu(ItHept)Cl]. An oven-dried flask equipped with a stir bar was charged with ItHept·HCl (30.1 mg, 0.1 mmol, 1.0 equiv), CuCl (19.8 mg, 0.2 mmol, 2.0 equiv) and K₂CO₃ (41.4 mg, 0.3 mmol, 3.0 equiv), placed under a positive pressure of argon, and subjected to three evacuation/backfilling cycles under high vacuum. 1,4-Dioxane (1.0 mL) was added, and the reaction mixture was stirred at 80° C. for 15 h. After the indicated time, the reaction mixture was diluted with CH₂Cl₂ (10 mL) and filtered. The solution was collected and concentrated. The title product was obtained by trituration from hexanes as a white solid. Yield 74% (26.9 mg). Mp=197-198° C. ¹H NMR (500 MHz, CDCl₃) δ7.01 (s, 2H), 2.19 (q, J=7.2 Hz, 12H), 0.69 (t, J=7.3 Hz, 18H). ¹³C NMR (126 MHz, CDCl₃) δ174.35, 118.04, 66.54, 30.34, 7.53. IR (KBr, v, cm⁻¹) 3127, 3097, 2969, 2944, 2878, 1614, 1567, 1466, 1452, 1376, 121.31173859, 758. HRMS (ESI) m/z: [2M−Cl]⁺ Calcd for C₃₄H₆₄N₄ClCu₂ 691.3389; Found 691.3389.

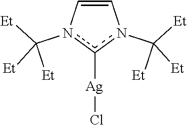

1,3-bis(3-ethylpentan-3-yl)-1H-imidazol-2-ylidene silver(I) chloride [Ag(ItHept)Cl]. An oven-dried flask equipped with a stir bar was charged with ItHept·HCl (30.1 mg, 0.1 mmol, 1.0 equiv), Ag₂O (46.3 mg, 0.2 mmol, 2.0 equiv) and K₂CO₃ (41.4 mg, 0.3 mmol, 3.0 equiv), placed under a positive pressure of argon, and subjected to three evacuation/backfilling cycles under high vacuum. 1,4-Dioxane (1.0 mL) was added, and the reaction mixture was stirred at 80° C. for 15 h. After the indicated time, the reaction mixture was diluted with CH₂Cl₂ (10 mL) and filtered. The solution was collected and concentrated. The title product was obtained by trituration from hexanes as a white solid (29.3 mg, 72% yield). Mp=201-202° C. ¹H NMR (500 MHz, CDCl₃) δ7.09 (d, J^{Ag}=2.2 Hz, 2H), 2.14 (q, J^{Ag}=7.3 Hz, 12H), 0.67 (t, J=7.4 Hz, 18H). ¹³C NMR (1.26 MHz, CDCl₃) δ177.24 (dd,J^{Ag}=158.3, 18.6 Hz), 118.55 (d, J^{Ag}=8.3 Hz), 66.72, 30.13, 7.50. IR (KBr, v, cm⁻¹) 3159, 3162, 3101, 2965, 2919, 2881, 1618, 1569, 1450, 1314, 1212, 1170, 857, 757. HRMS (ESI) m/z: [2M−Cl]⁺ Calcd for C₃₄H₆₄N₄ClAg₂ 779.2911; Found 779.2937.

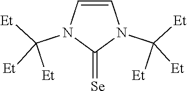 [SMILES: CCC(CC)(CC)n1ccn(C(CC)(CC)CC)c1=[Se]]

1,3-bis(3-ethylpentan-3-yl)-1,3-dihydro-2Hanidazole-2-selenone [Se(ItHept)]. An oven-dried flask equipped with a stir bar was charged with ItHept·HCl (30.1 mg, 0.1 mmol, 1.0 equiv), selenium (15.8 mg, 0.2 mmol, 2.0 equiv), and K₂CO₃ (41.4 mg, 0.3 mmol, 3.0 equiv), placed under a positive pressure of argon, and subjected to three evacuation/backfilling cycles under high vacuum. 1,4-Dioxane (1.0 mL) was added, and the reaction mixture was stirred at 80° C. for 15 h. After the indicated time, the reaction mixture was diluted with CH₂Cl₂ (10 mL) and filtered. The solution was collected and concentrated. The title product was obtained by trituration from hexanes as a white solid (28.5 mg, 83% yield). Mp=164-165° C. ¹H NMR (500 MHz, CDCl₃) δ6.98 (s, 2H), 2.48 (q, J=6.8 Hz, 12H), 0.71 (t, J=7.4 Hz, 18H). ¹³C NMR (126 MHz, CDCl₃) δ150.63, 117.74, 70.34, 24.90, 7.97. ⁷⁷Se NMR (95 MHz, CDCl₃) δ180.59. IR (KBr, v, cm⁻¹) 3202, 3168, 2957, 2891, 2874, 1580, 1550, 1456, 1344, 1309, 1211, 1162, 851, 712, 686. HRMS (ESI) m/z: [2M+H]⁺ Calcd for C₃₄H₆₅N₄Se₂ 687.3544; Found 687.3515.

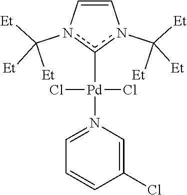

3-chloropyridine [1,3-bis(3-ethylpentan-3-yl)-1H-imidazol-2-ylidene] palladium(II) dichloride [Pd(ItHept)(3ClPy)Cl₂]. An oven-dried flask equipped with a stir bar was charged with ItHept·HCl (30.1 mg, 0.1 mmol, 1.0 equiv), PdCl₂ (17.7 mg, 0.1 mmol, 1.0 equiv) and K₂CO₃ (41.4 mg, 0.3 mmol, 3.0 equiv), placed under a positive pressure of argon and subjected to three evacuation/backfilling cycles under high vacuum. 3-Chloropytidine (0.5 mL) was added, and the reaction mixture was stirred at 80° C. for 15 h. After the indicated time, the reaction mixture was diluted with CH₂Cl₂ (10 mL) and filtered. The solution was collected and concentrated. The title product was obtained by trituration from hexanes as a yellow solid (41.7 mg, 75% yield). Mp=228-229° C. ¹H NMR (500 MHz, CDCl₃) δ8.99 (d, J=2.1 Hz, 1H), 8.93 (d,J=5.4 Hz, 1H), 7.74 (d,J=8.2 Hz, 1H), 7.29 (dd,J=8.2, 5.5 HZ, 1H), 7.16 (s, 2H), 2.70 (q, J=7.1 Hz, 12H), 0.86 (t, J=7.3 Hz, 18H). ¹³C NMR (126 MHz, CDCl₃) δ151.02, 150.08, 142.74, 137.95, 132.77, 125.06, 120.76, 69.99, 30.32, 8.69. IR (KBr, v, cm⁻¹) 3147, 3128, 3094, 2965, 2879, 1594, 1560, 1463, 1214, 1160, 1118. 1050, 854, 805, 688. HRMS (ESI) m/z: [M−Cl]⁺ Calcd for C₂₂H₃₆N₃Cl₂Pd 518.1318; Found 518.1344.

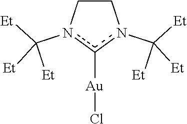

1,3-bis(3-ethylpentan-3-yl)-4,5-dihydro-1H-imidazol-2-ylidene gold(I) chloride [Au(SItHept)Cl]. An oven-dried flask equipped with a stir bar was charged with SItHept·HCl (36.4 mg, 0.12 mmol, 1.2 equiv), placed under a positive pressure of argon and subjected to three evacuation/backfilling cycles under high vacuum. THF (0.5 mL) and LiHMDS (1.0 M in THF, 0.11 mL, 0.11 mmol, 1.1 equiv) were added and the reaction mixture was stirred at room temperature for 2 h. After the indicated time, a solution of AuClSMe₂ (29.5 mg, 0.1 mmol, 1.0 equiv) in THF (0.5 mL) was added dropwise, and the resulting mixture was stirred at room temperature for 15 h. After the indicated time, the reaction mixture was diluted with CH₂Cl₂ (10 mL) and filtered. The solution was collected and concentrated. The title product was obtained by trituration from hexanes as a white solid (33.9 mg, 68% yield). Mp=120-121° C. ¹H NMR (500 MHZ, CDCl₃) δ3.59 (s, 4H), 2.19 (q, J=7.3 Hz, 12H), 0.80 (t, J=7.4 Hz, 18H). ¹³C NMR (126 MHz, CDCl₃) δ192.22, 65.24, 48.26, 29.31, 7.96. IR (KBr, v, cm⁻¹) 2966, 2920, 2880, 1423, 1264, 1033. HRMS (ESI) m/z: [2M−Cl]⁺ Calcd for C₃₄H₆₈N₄ClAu₂ 961.4458; Found 961.4474.

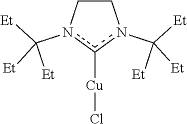

1,3-bis(3-ethylpentan-3-yl)-4,5-dihydro-1H-imidazol-2-ylidene copper(I) chloride [Cu(SItHept)Cl]. An oven-dried flask equipped with a stir bar was charged with SItHept·HCl (30.3 mg, 0.1 mmol, 1.0 equiv), CuCl (19.8 mg, 0.2 mmol, 2.0 equiv), and KO^{t}Bu (33.7 mg, 0.3 mmol, 3.0 equiv), placed under a positive pressure of argon, and subjected to three evacuation/backfilling cycles under high vacuum. THF (1.0 mL) was added, and the reaction mixture was stirred at room temperature for 15 h. After the indicated time, the reaction mixture was diluted with CH₂Cl₂ (10 mL) and filtered. The solution was collected and concentrated. The title product was obtained by trituration from hexanes as a white solid (16.6 mg, 55% yield). Mp=146-147° C. ¹H NMR (500 MHz, CDCl₃) δ3.47 (s, 4H), 1.93 (q, J=7.2 Hz, 12H), 0.77 (t, J=7.3 Hz, 18H). ¹³C NMR (126 MHZ, CDCl₃) δ198.07, 63.59, 46.72, 29.21, 7.83. IR (KBr, v, cm1) 2967, 2880, 1456, 1425, 1333, 1262, 1154. HRMS (ESI) m/z: [2M−Cl]⁺ Calcd for C₃₄H₆₈N₄ClCu₂ 695.3702; Found 695.3681.

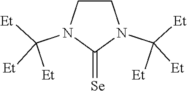 [SMILES: CCC(CC)(CC)N1CCN(C(CC)(CC)CC)C1=[Se]]

1,3-bis(3-ethylpentan-3-yl)imidazolidine-2-selenone [Se(SItHept)]. An oven-dried flask equipped with a stir bar was charged with SItHept·HCl (30.3 mg, 0.1 mmol, 1.0 equiv), selenium (15.8 mg, 0.2 mmol, 2.0 equiv), and KO^{t}Bu (33.7 mg, 0.3 mmol, 3.0 equiv), placed under a positive pressure of argon, and subjected to three evacuation/backfilling cycles under high vacuum. THF (1.0 mL) was added, and the reaction mixture was stirred at room temperature for 15 h. After the indicated time, the reaction mixture was diluted with CH₂Cl₂ (10 mL) and filtered. The solution was collected and concentrated. The title product was obtained by trituration from hexanes as a white solid (29.4 mg, 85% yield). Mp=146-147° C. ¹H NMR (500 MHz, CDCl₃) δ3.53 (s, 4H), 2.28 (q, J=6.8 Hz, 12H), 0.81 (t,J=7.4 Hz, 18H). ¹³C NMR (126 MHz, CDCl₃) δ177.44, 67.04, 48.33, 25.54, 8.40. ⁷⁷Se NMR (95 MHz, CDCl₃) δ246.62. IR (KBr, v, cm1) 2959, 2872, 1454, 1395, 1273, 1246, 1214, 1149. HRMS (ESI) m/z: [M+K]⁺ Calcd for C₁₇H₃₄N₂SeK 385.1519; Found 385.1534.

25

### Example 3: Characterization of NHC Ligands and NHC Catalyst Complexes

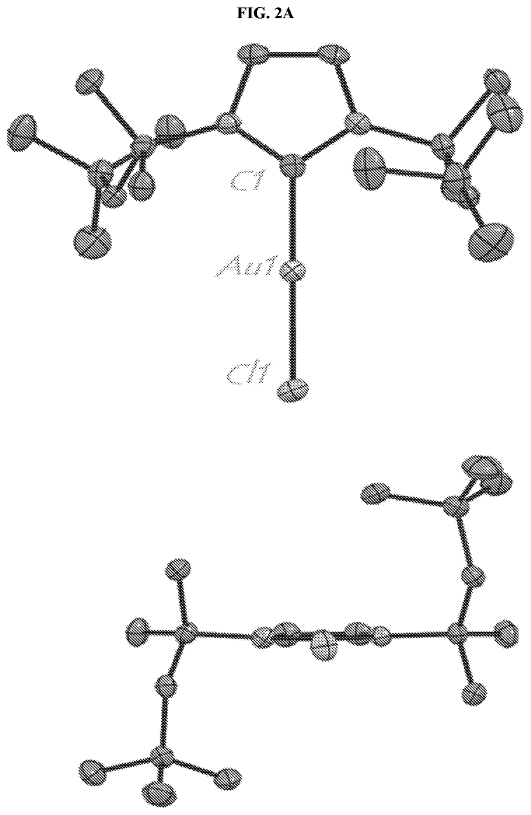

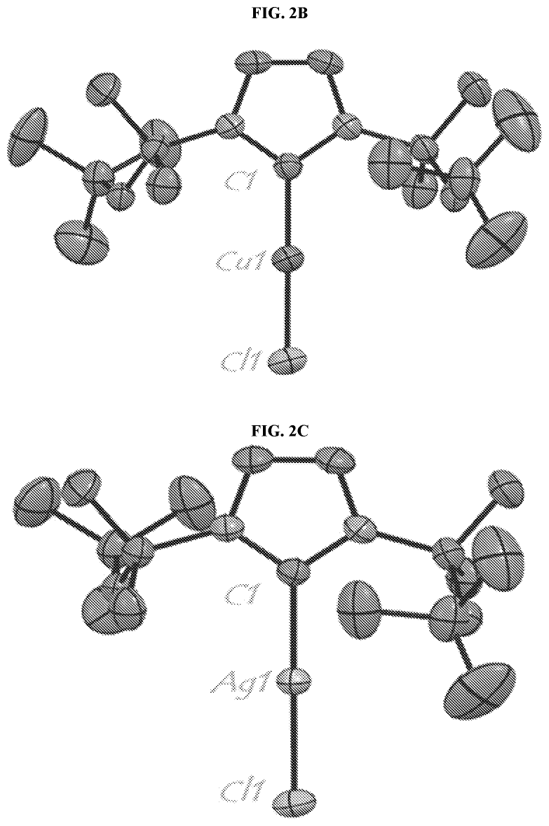

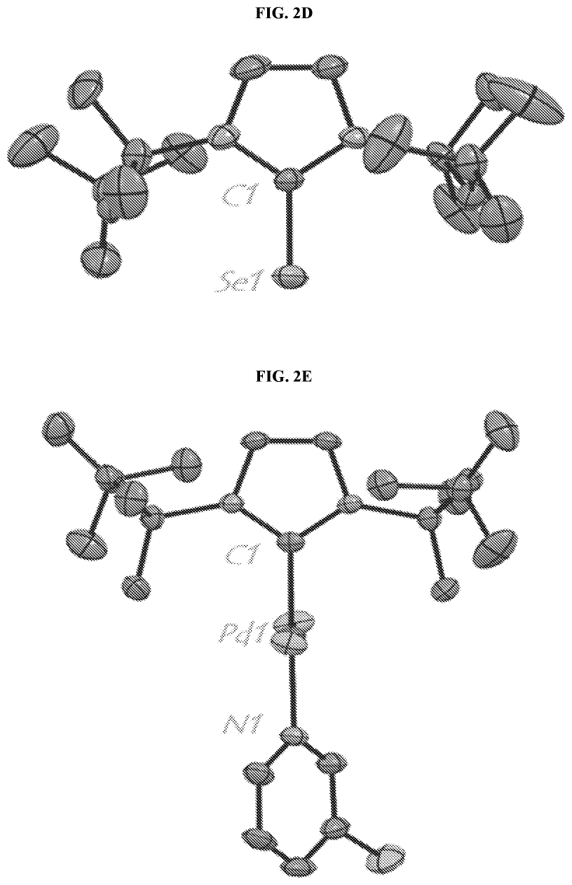

NHC complexes [Ag(ItOct)Cl], [Cu(ItOct)Cl], [Se(ItOct)], and [Pd(ItOct)(3-py)Cl₂] were fully characterized by x-ray crystallography (FIGS. 2A-2E). Importantly, the buried volume (% V_{bur}) of linear [Ag(ItOct)Cl], [Cu(ItOct)Cl], and [Se(ItOct)] complexes was found to be 43.7%, 48.2% and 44.1%, which confirms the significant steric impact of the ItOct substitution. In contrast, (% V_{bur}) of square planar [Pd(ItOct)(3-py)Cl₂] (14) is much lower (38.4%). Without wishing to be bound by theory, this demonstrates the capacity of the tert-octyl side chains to adjust to the steric impact of the metal center.

The selenourea adduct [Se(ItOct)] permitted assessment of π-backbonding of ItOct from the 77Se NMR spectra. The δ_{Se} value of 216.7 ppm for [Se(ItOct)] (CDCl₃) suggests that ItOct is more π-accepting than ItBu (δ_{Se}, 183 ppm, CDCl₃). Furthermore, 1J_{CH} coupling constant from the ¹³C satellites of ¹H NMR spectra of 219.60 Hz for ItOct·HCl (CDCl₃) gives an accurate indication of σ-donation, and is consistent with this ligand as more strongly donating than N-aryl ligands, such as IPr(1J_{CH}=223.70 Hz; cf. ItBu: 1J_{CH}=219.35 Hz).

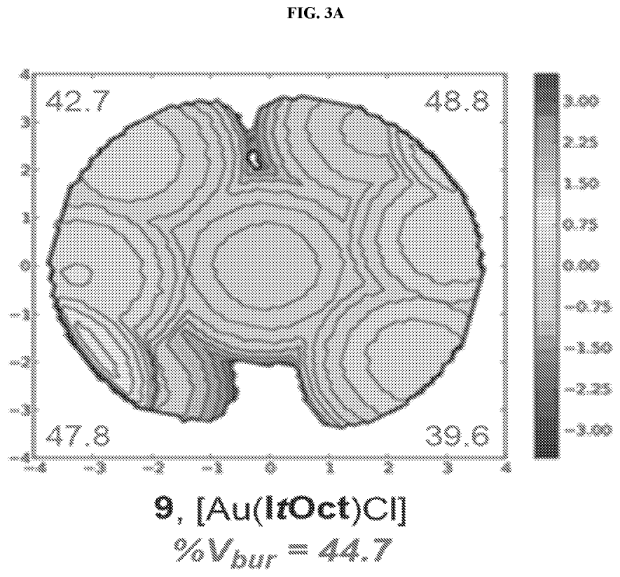

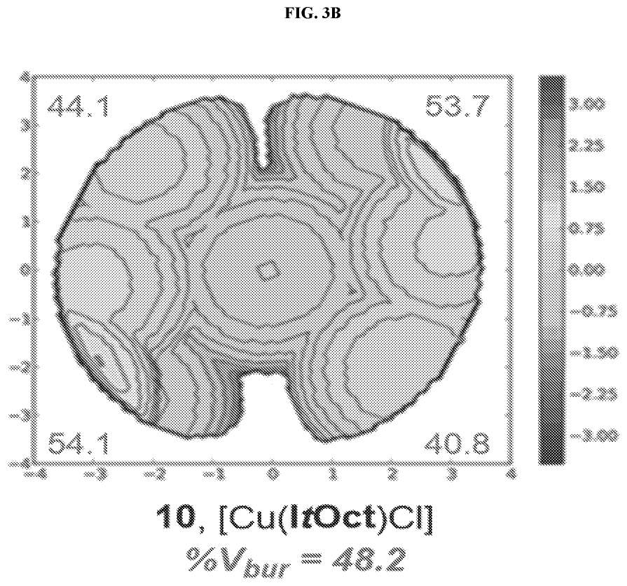

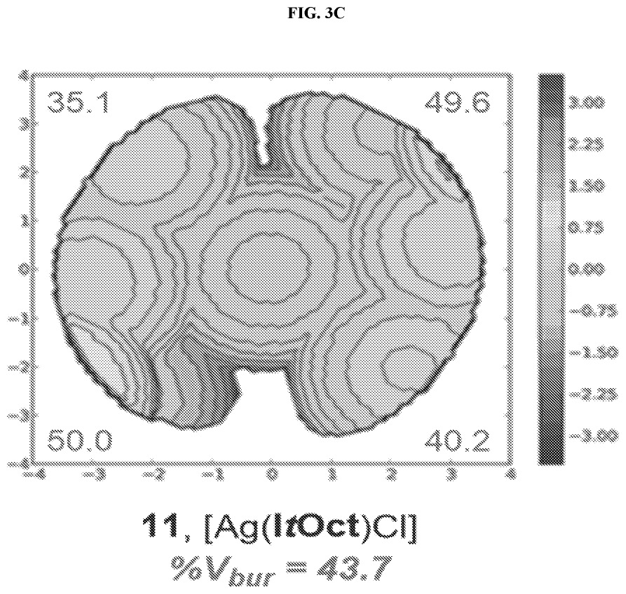

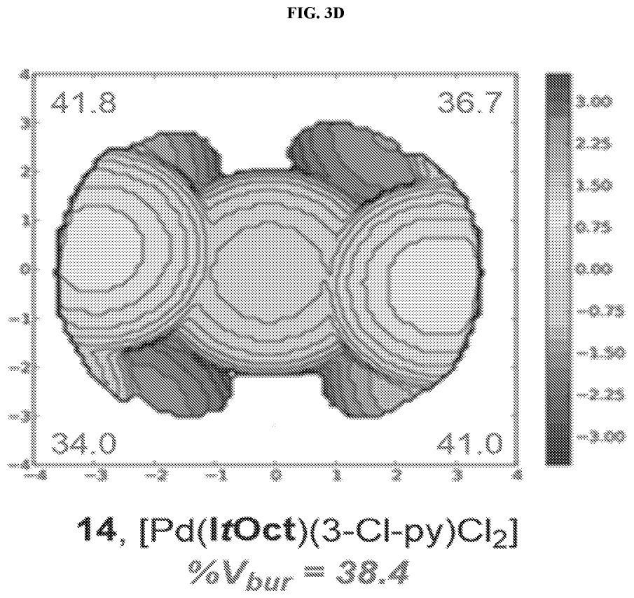

The x-ray structure of [Au(ItOct)Cl] revealed a C₂-symmeric Z-shape arrangement of N-alkyl substituents with a linear (C—Au—Cl, 179.9°; C—Au, 2.007 Å) geometry. The % buried volume (% V_{bur}) of [Au(ItOct)Cl] is 44.7%. Thus, [Au(ItOct)Cl] represents the most bulky N-alkyl NHC ligand prepared to date. This value can be compared with the (% V_{bur}) of 39.6% determined for [Au(ItBu)Cl]. Furthermore, the gem-dimethyl substitution of the longer tert-Octyl side-chain places the metal within the pocket formed by the alkyl side chain. The steric mapping of various metal centers, including [Au(ItOct)Cl], are shown in FIGS. 3A-3C.

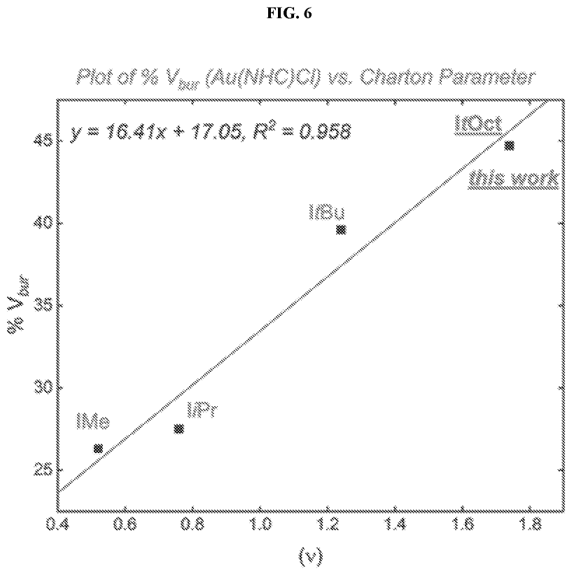

Interestingly, a strong linear correlation between the % buried volume (% V_{bur}) and the steric Charton parameter (n) (FIG. 6) was observed using linear [Au(NHC)Cl] complexes. This finding further establishes ItOct as the most sterically-demanding N-alkyl-substituted NHC ligand. Finally, the expensive yet extremely useful bulky adamantyl (IAd) is much smaller in volume than t-octyl (% V_{bur}=39.8%, IAd vs. 44.7%, ItOct).

### Example 4: Catalytic Activity of NHC Catalyst Complexes

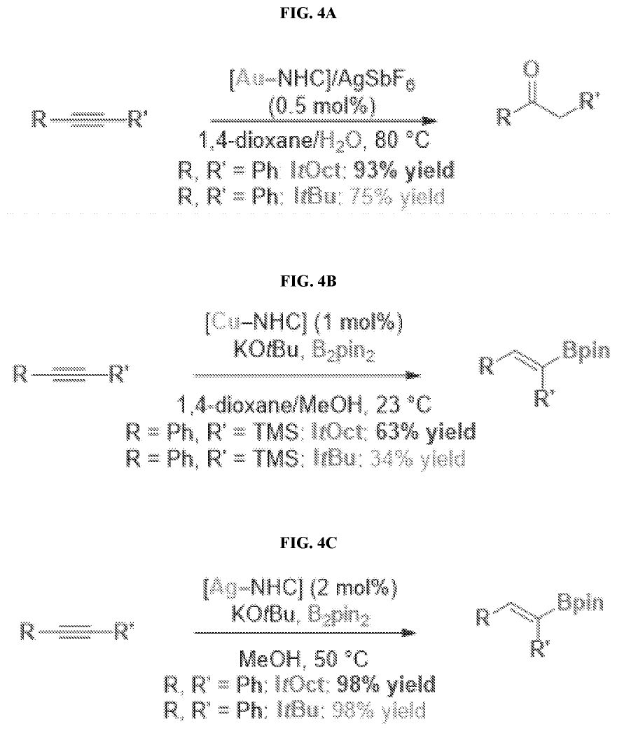

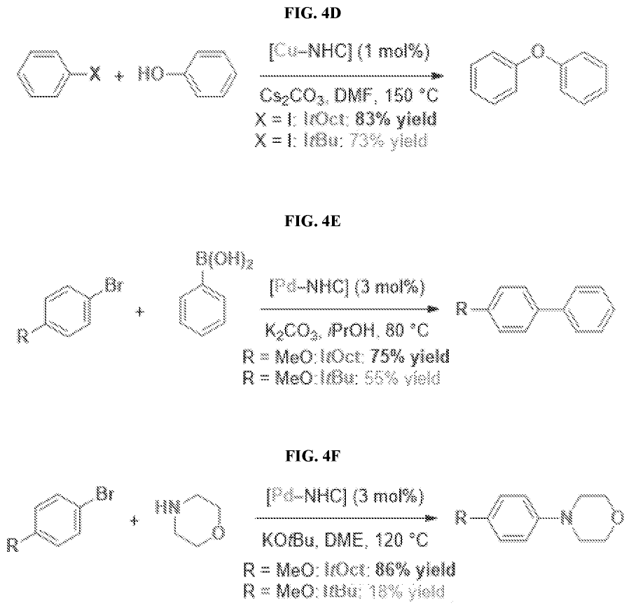

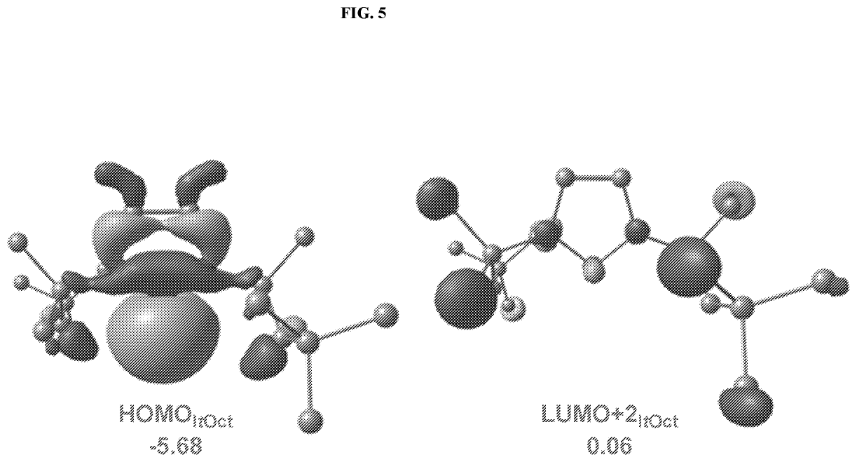

The increased steric bulk of the ItOct (1) ligand was envisaged to be beneficial for transition-metal catalytic reactions. To demonstrate the effect of increased steric substitution, several representative reactions were performed employing the Au, Ag, Cu, and Pd NHC catalyst complexes of the present disclosure, including [Au(ItOct)Cl], [Cu(ItOct)Cl], [Ag(ItOct)Cl], and [Pd(ItOct)(3-Cl-py)Cl₂]. The performance of the aforementioned NHC catalyst complexes were evaluated in Au-catalyzed hydration, Cu-catalyzed hydroboration, Ag-catalyzed hydroboration, Cu-catalyzed C—O coupling, and Pd-catalyzed C—C and C—N coupling, wherein the results each compare favorably with the results observed with the analogous [M(ItBu)X] catalyst (FIGS. 4A-4F).

### Catalytic Activity of NHC Catalyst Complexes

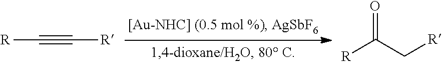

[Au—NHC]-Catalyzed Hydration of Alkynes. An oven-dried vial equipped with a stir bar was charged with an alkyne substrate (1.0 mmol), [Au—NHC] catalyst (0.5 mol %), AgSbF₆ (catalytic), 1,4-dioxane (1.0 mL) and stirred for 1 min. Distilled water (0.1 mL) was added and the reaction mixture was stirred at 80° C. for 15 h. After the indicated time, the reaction mixture was diluted with water (5 mL), extracted with CH₂Cl₂ (3×5 mL), dried and concentrated. The sample was analyzed by ¹HNMR (CDCl₃, 500 MHz) and GC-MS to obtain conversion, selectivity and yield using internal standard and comparison with authentic samples. Crude material purified by column chromatography.

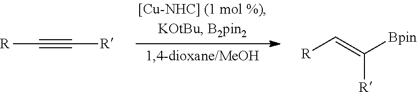

[Cu—NHC]-Catalyzed Hydroboration of Alkynes. An oven-dried vial equipped with a stir bar was charged with an alkyne substrate (1.0 mmol, 1.0 equiv), bis(pinacolato)diboron (B₂pin₂) (304.7 mg, 1.2 mmol, 1.2 equiv), KOtBu (11.2 mg, 0.1 mmol, 0.1 equiv), 1,4-dioxane (2.4 mL), MeOH (0.2 mL), [Cu—NHC]-catalyst (1.0 mol %) and stirred at room temperature for 15 h. After the indicate time, the solvent was removed under reduce pressure. The sample was analyzed by ¹H NMR (CDCl₃, 500 MHz) and GC-MS to obtain conversion, selectivity and yield using internal standard and comparison with authentic samples. Crude material was purified by column chromatography.

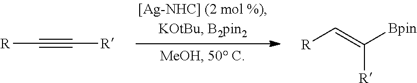

[Ag·NHC]-Catalyzed Hydroboration of Alkynes. An oven-dried vial equipped with a stir bar was charged with an alkyne substrate (1.0 mmol, 1.0 equiv), bis(pinacolato)diboron (B₂pin₂) (380.9 mg, 1.5 mmol, 1.5 equiv), KOtBu (11.2 mg, 0.1 mmol, 0.1 equiv), 1,4-dioxane (2.4 mL), and [Ag—NHC]-catalyst (2.0 mol %), placed under a positive pressure of argon, and subjected to three evacuation/backfilling cycles under high vacuum. MeOH (2.0 mL) was added with vigorous stirring and the reaction mixture was stirred at 50° C. for 15 h. After the indicated time, the solvent was removed under reduced pressure. The same was analyzed by ¹H NMR (CDCl₃, 500 MHz) and GC-MS to obtain conversion, selectivity and yield using an internal standard and comparison with authentic samples. Crude material was purified by column chromatography.

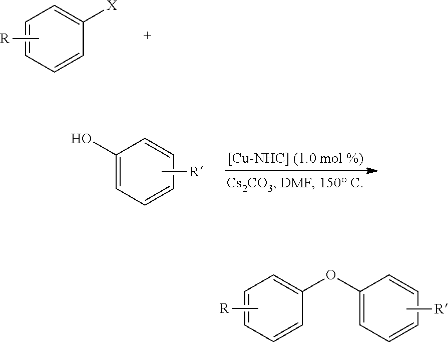

[Cu—NHC]-Catalyzed C-0 Cross-Coupling. An oven dried vial equipped with a stir bar was charged with a phenol (47.1 mg, 0.50 mmol, 1.0 equiv), aryl halide (0.50 mmol, 1.0 equiv), Cs₂CO₃ (244.4 mg, 0.75 mmol, 1.5 equiv), [Cu—NHC] catalyst (1.0 mol %), and DMF (1.0 mL), and stirred at 150° C. for 15 h. After the indicated time, the reaction mixture was cooled down to room temperature, diluted with CH₂Cl₂ (10 mL), filtered and concentrated. The sample was analyzed by 1HNMR (CDCl₃, 500 MHz) and GC-MS to obtain conversion, selectivity and yield using internal standard and comparison with authentic samples. Crude material was purified by column chromatography.

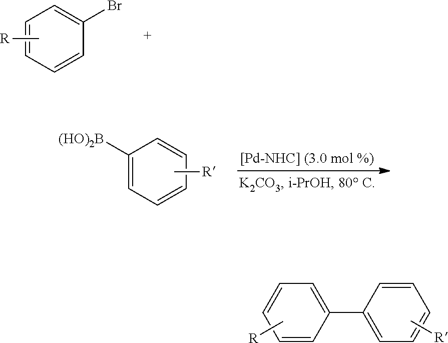

[Pd—NHC]-Catalyzed Suzuki Cross-Coupling. An oven dried vial equipped with a stir bar was charged with an aryl bromide substrate (0.2 mmol, 1.0 equiv), boronic acid (0.4 mmol, 2.0 equiv), K₂CO₃ (82.8 mg, 0.6 mmol, 3.0 equiv), [Pd—NHC] catalyst (3.0 mol %), placed under a positive pressure of argon, and subjected to three evacuation/backfilling cycles under high vacuum. i-PrOH (0.8 mL) was added and the reaction mixture was stirred at 80° C. for 15 h. After the indicated time, the reaction mixture was diluted with CH₂Cl₂ (10 mL), filtered, and concentrated. The sample was analyzed by 1HNMR (CDCl₃, 500 MHz) and GC-MS to obtain conversion, selectivity and yield using internal standard and comparison with authentic samples. Crude material was purified by column chromatography.

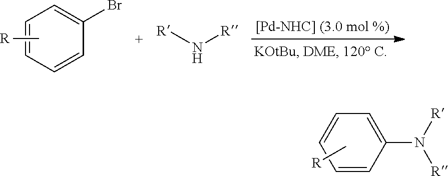

[Pd—NHC]-Catalyzed Buchwald-Hartwig Cross-Coupling. An oven dried vial equipped with a stir bar was charged with an aryl bromide substrate (0.2 mmol, 1.0 equiv), morpholine (34.8 mg, 0.4 mmol, 2.0 equiv), KOtBu (56.1 mg, 0.5 mmol, 2.5 equiv), and [Pd—NHC] catalyst (3 mol %), then placed under a positive pressure of argon, and subjected to three evacuation/backfilling cycles under high vacuum. DME (1.0 mL) was added and the reaction mixture was stirred at 120° C. for 15 h. After the indicated time, the reaction mixture was diluted with CH₂Cl₂ (10 mL), washed with water (10 mL), extracted with CH₂Cl₂ (2×10 mL), dried and concentrated. The sample was analyzed by 1HNMR (CDCl₃, 500 MHz) and GC-MS to obtain conversion, selectivity and yield using internal standard and comparison with authentic samples.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### Enumerated Embodiments

The following exemplary embodiments are provided, the numbering of which is not to be construed as designating levels of importance:

Embodiment 1 provides a compound of formula (I):

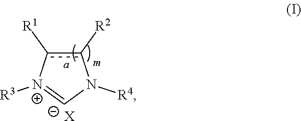

wherein:
X is a counter anion;
R¹ and R² are each independently selected from the group consisting of H, optionally substituted C₁-C₆ alkyl, optionally substituted C₂-C₆ alkenyl, optionally substituted benzyl, optionally substituted phenyl, and optionally substituted naphthyl, and optionally substituted C₄-C₁₀ heteroaryl, or
   R¹ and R² may combine with the ring to which they are attached to form C₅-C₁₂ cycloalkyl, C₆-C₁₀ aryl, or C₄-C₁₀ heteroaryl,
   wherein each optional substituent in R¹ and R² is independently at least one substituent selected from the group consisting of C₁-C₃ alkyl, C₂-C₆ alkenyl, phenyl, naphthyl, C₄-C₁₀ heteroaryl, N(R^{a})(R^{b}), OR^{b}, CN, CF₃, OCF₃, C(═O)R^{b}, N(R)S(═O)₂R^{b}, C(═O)N(R^{a})(R^{b}), and C(═O)OR^{b};
R³ and R⁴ are each independently

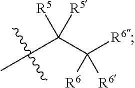

R⁵ and R^{5′} are each independently selected from the group consisting of C₁-C₆ alkyl, C₃-C₁₂ cycloalkyl, C₂-C₁₀ heterocycloalkyl, C₆-C₁₀ aryl, C₄-C₁₀ heteroaryl, halogen, OR^{b}, N(R^{a})(R^{b}), SR^{b}, and Si(R^{b})₃,
   wherein R⁵ and R^{5′} may combine with the carbon atom to which they are bound to form one selected from the group consisting of C₃-C₈ cycloalkyl and C₂-C₇ heterocycloalkyl;
R⁶, R^{6′}, and R^{6″} are each independently selected from the group consisting of H, C₁-C₆ alkyl, C₃-C₁₂ cycloalkyl, C₂-C₁₀ heterocycloalkyl, C₆-C₁₀ aryl, C₄-C₁₀ heteroaryl, halogen, OR^{b}, N(R^{a})(R^{b}), SR^{b}, and Si(R^{b})₃,
   wherein at least two of R⁶, R^{6′}, and R^{6″} may combine with the carbon atom to which they are bound to form one selected from the group consisting of C₃-C₁₂ cycloalkyl, C₂-C₁₀ heterocycloalkyl, C₅-C₁₂ fused cycloalkyl, C₄-C₁₀ fused heterocycloalkyl, C₅-C₁₂ bridged cycloalkyl, and C₄-C₁₀ bridged heterocycloalkyl,
   wherein any one of R⁶, R^{6′}, and R^{6″} may combine with any one of R⁵ and R^{5′} and the carbon atoms to which they are bound to form one selected from the group consisting of C₄-C₁₂ cycloalkyl and C₃-C₁₀ heterocycloalkyl, and
   wherein if R⁵ and R^{5′} are Me, and two of R⁶, R^{6′}, and R^{6″} are H, then the other one of R⁶, R^{6′}, and R^{6″} is not H or Me;
   wherein if R⁵ and R^{5′} are Me, two of R⁶, R^{6′}, and R^{6″} are H, and one of R⁶, R^{6′}, and R^{6″} is t-Bu, then M is not BF₄;
bond a is a single or double bond;
each occurrence of R^{a} is independently H or C₁-C₃ alkyl;
each occurrence of R^{b} is independently selected from the group consisting of H, C₁-C₆ alkyl, C₁-C₃ haloalkyl, C₂-C₆ alkenyl, benzyl, naphthyl, C₄-C₁₀ heteroaryl, and phenyl optionally substituted with at least one substituent selected from the group consisting of C₁-C₃ alkyl and halogen; and
m is 1 or 2.

Embodiment 2 provides the compound of Embodiment 1, wherein X is selected from the group consisting of H, halide, OS(═O)₂R^{b}, OC(═O)R^{b}, N(C(═O)R^{b})₂, tetracoordinate boronate, and hexacoordinate phosphorus.

Embodiment 3 provides the compound of Embodiment 2, wherein X is Cl or BF₄.

Embodiment 4 provides the compound of any one of Embodiments 1-3, wherein at least one of R⁵ and R^{5′} is Me or Et.

Embodiment 5 provides the compound of any one of Embodiments 1-4, wherein two of R⁶, R^{6′}, and R^{6″} are H.

Embodiment 6 provides the compound of any one of Embodiments 1-5, wherein at least one of R⁶, R^{6′}, and R^{6″} is tert-butyl or Me.

Embodiment 7 provides the compound of any one of Embodiments 1-6, wherein at least one of R¹ and R² is H or Me.

Embodiment 8 provides the compound of any one of Embodiments 1-7, wherein R¹ and R² are identical.

Embodiment 9 provides the compound of any one of Embodiments 1-8, wherein at least one of R³ and R⁴ is

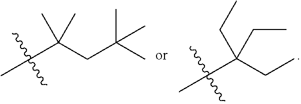

Embodiment 10 provides the compound of any one of Embodiments 1-9, wherein R³ and R⁴ are identical.

Embodiment 11 provides the compound of any one of Embodiments 1-10, wherein m is 1.

Embodiment 12 provides the compound of Embodiment 11, which is selected from the group consisting of:
1,3-bis(2,4,4-trimethylpentan-2-yl)-1H-imidazol-3-ium chloride;
1,3-bis(2,4,4-trimethylpentan-2-yl)-1H-imidazol-3-ium tetrafluoroborate;
1,3-bis(2,4,4-trimethylpentan-2-yl)-4,5-dihydro-1H-imidazol-3-ium chloride;
1,3-bis(2,4,4-trimethylpentan-2-yl)-1H-benzo[d]imidazol-3-ium chloride;
1,3-bis(3-ethylpentan-3-yl)-1H-imidazol-3-ium chloride; and
1,3-bis(3-ethylpentan-3-yl)-4,5-dihydro-1H-imidazolium chloride.

Embodiment 13 provides a compound of formula (II):

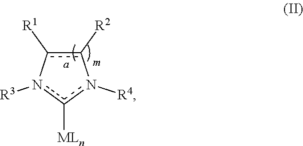

wherein:
M is a transition metal;
L is a ligand of M, wherein each occurrence of L can be the same or different;
R¹ and R² are each independently selected from the group consisting of H, optionally substituted C₁-C₆ alkyl, optionally substituted C₂-C₆ alkenyl, optionally substituted benzyl, optionally substituted phenyl, and optionally substituted naphthyl, and optionally substituted C₄-C₁₀ heteroaryl, or
   R¹ and R² may combine with the ring to which they are attached to form C₅-C₁₂ cycloalkyl, C₆-C₁₀ aryl, or C₄-C₁₀ heteroaryl,
   wherein each optional substituent in R¹ and R² is independently at least one substituent selected from the group consisting of C₁-C₃ alkyl, C₂-C₆ alkenyl, phenyl, naphthyl, C₄-C₁₀ heteroaryl, N(R^{a})(R^{b}), OR^{b}, CN, CF₃, OCF₃, C(═O)R^{b}, N(R)S(═O)₂R^{b}, C(═O)N(R^{a})(R^{b}), and C(═O)OR^{b};
R³ and R⁴ are each independently

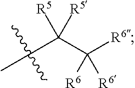

R⁵ and R^{5′} are each independently selected from the group consisting of C₁-C₆ alkyl, C₃-C₁₂ cycloalkyl, C₂-C₁₀ heterocycloalkyl, C₆-C₁₀ aryl, C₄-C₁₀ heteroaryl, halogen, OR^{b}, N(R^{a})(R^{b}), SR^{b}, and Si(R^{b})₃,
   wherein R⁵ and R^{5′} may combine with the carbon atom to which they are bound to form one selected from the group consisting of C₃-C₈ cycloalkyl and C₂-C₇ heterocycloalkyl;
R⁶, R^{6′}, and R^{6″} are each independently selected from the group consisting of H, C₁-C₆ alkyl, C₃-C₁₂ cycloalkyl, C₂-C₁₀ heterocycloalkyl, C₆-C₁₀ aryl, C₄-C₁₀ heteroaryl, halogen, OR^{b}, N(R^{a})(R^{b}), SR^{b}, and Si(R^{b})₃,
   wherein at least two of R⁶, R^{6′}, and R^{6″} may combine with carbon atom to which they are bound to form one selected from the group consisting of C₃-C₁₂ cycloalkyl, C₂-C₁₀ heterocycloalkyl, C₅-C₁₂ fused cycloalkyl, C₄-C₁₀ fused heterocycloalkyl, C₅-C₁₂ bridged cycloalkyl, and C₄-C₁₀ bridged heterocycloalkyl,
   wherein any one of R⁶, R^{6′}, and R^{6″} may combine with any one of R⁵ and R^{5′} and the carbon atoms to which they are bound to form one selected from the group consisting of C₄-C₁₂ cycloalkyl and C₃-C₁₀ heterocycloalkyl, and
   wherein if R⁵ and R^{5′} are Me, and two of R⁶, R^{6′}, and R^{6″} are H, then the other one of R⁶, R^{6′}, and R^{6″} is not H or Me;
each occurrence of [symbol] is a single or double bond,
   wherein no more than one [symbol] bonding a C atom and a N atom is a double bond;
each occurrence of R^{a} is independently H or C₁-C₃ alkyl;
each occurrence of R^{b} is independently selected from the group consisting of H, C₁-C₆ alkyl, C₁-C₃ haloalkyl, C₂-C₆ alkenyl, benzyl, naphthyl, C₄-C₁₀ heteroaryl, and phenyl optionally substituted with at least one substituent selected from the group consisting of C₁-C₃ alkyl and halogen;
n is an integer which is selected from the group consisting of 1, 2, 3, 4, and 5; and
m is an integer which is either 1 or 2.

Embodiment 14 provides the compound of Embodiment 13, wherein M is selected from the group consisting of Cu, Ag, Au, Pd, Ni, Pt, Co, Rh, Ir, Fe, Ru, and Os.

Embodiment 15 provides the compound of Embodiment 13 or 14, wherein L is an anionic ligand or a neutral ligand.

Embodiment 16 provides the compound of Embodiment 15, wherein the anionic ligand is selected from the group consisting of H, OS(═O)₂R^{b}, OC(═O)R^{b}, N(C(═O)R^{b})₂, halogen, C₆-C₁₀ aryl, and C₄-C₁₀ heteroaryl.

Embodiment 17 provides the compound of Embodiment 16, wherein the anionic ligand is Cl.

Embodiment 18 provides the compound of Embodiment 15, wherein the neutral ligand is pyridyl optionally substituted with at least one halogen or a C₂-C₁₂ alkene.

Embodiment 19 provides the compound of Embodiment 18, wherein the neutral ligand is

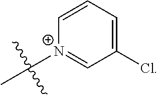 [SMILES: *[n+]1cccc(Cl)c1]

Embodiment 20 provides the compound of any one of Embodiments 13-19, wherein at least one of R⁵ and R^{5′} is Me or Et.

Embodiment 21 provides the compound of any one of Embodiments 13-20, wherein two of R⁶, R^{6′}, and R^{6″} are H.

Embodiment 22 provides the compound of any one of Embodiments 13-21, wherein at least one of R⁶, R^{6′}, and R^{6″} is tert-butyl or Me.

Embodiment 23 provides the compound of any one of Embodiments 13-22, wherein at least one of R¹ and R² is H or Me.

Embodiment 24 provides the compound of any one of Embodiments 13-23, wherein R¹ and R² are identical.

Embodiment 25 provides the compound of any one of Embodiments 13-24, wherein at least one of R³ and R⁴ is

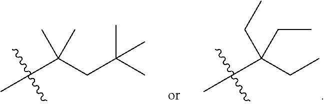

Embodiment 26 provides the compound of any one of Embodiments 13-25, wherein R³ and R⁴ are identical.

Embodiment 27 provides the compound of any one of Embodiments 13-26, wherein m is 1.

Embodiment 28 provides the compound of Embodiment 13, which is selected from the group consisting of:
1,3-bis(2,4,4-trimethylpentan-2-yl)-2,3-dihydro-1H-imidazol-2-ylidene gold(I) chloride;
1,3-bis(2,4,4-trimethylpentan-2-yl)-2,3-dihydro-1H-imidazol-2-ylidene copper(I) chloride;
1,3-bis(2,4,4-trimethylpentan-2-yl)-2,3-dihydro-1H-imidazol-2-ylidene silver(I) chloride;
1,3-bis(2,4,4-trimethylpentan-2-yl)-2,3-dihydro-1H-imidazol-2-ylidene palladium(II)(allyl) chloride;
3-chloropyridine [1,3-bis(2,4,4-trimethylpentan-2-yl)-2,3-dihydro-1H-imidazol-2-ylidene] palladium(II) dichloride;
1,3-bis(2,4,4-trimethylpentan-2-yl)imidazolidin-2-ylidene gold(I) chloride;
1,3-bis(2,4,4-trimethylpentan-2-yl)imidazolidin-2-ylidene palladium(II) (allyl) chloride;
1,3-bis(2,4,4-trimethylpentan-2-yl)benzimidazolidin-2-ylidene gold(I) chloride;
1,3-bis(2,4,4-trimethylpentan-2-yl)benzimidazolidin-2-ylidene copper(I) chloride;
3-chloropyridine [1,3-bis(2,4,4-trimethylpentan-2-yl)-benzimidazol-2-ylidene] palladium(II) dichloride
allyl [1,3-bis(2,4,4-trimethylpentan-2-yl)benzimidazolidin-2-ylidene] palladium(II) chloride;
1,3-bis(3-ethylpentan-3-yl)-1H-imidazol-2-ylidene gold(I) chloride;
1,3-bis(3-ethylpentan-3-yl)-1H-imidazol-2-ylidene copper(I) chloride;
1,3-bis(3-ethylpentan-3-yl)-1H-imidazol-2-ylidene silver(I) chloride;
3-chloropyridine [1,3-bis(3-ethylpentan-3-yl)-1H-imidazol-2-ylidene] palladium(II) dichloride;
1,3-bis(3-ethylpentan-3-yl)-4,5-dihydro-1H-imidazol-2-ylidene gold(I) chloride; and
1,3-bis(3-ethylpentan-3-yl)-4,5-dihydro-1H-imidazol-2-ylidene copper(I) chloride.

Embodiment 29 provides a method of making the compound of any one of Embodiments 1-12, the method comprising: contacting a compound of formula (III):

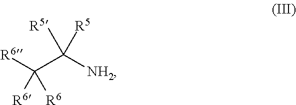

with (CHO)₂ to form a compound of formula (IV):

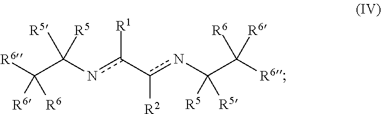

and cyclizing the compound of formula (IV) to form the compound of formula (I).

Embodiment 30 provides the method of Embodiment 29, wherein R³ and R⁴ in the compound of formula (I) are identical.

Embodiment 31 provides the method of Embodiment 29, wherein the compound of formula (IV) is treated with NaBH₄ to form a diamine compound of formula (IVb):

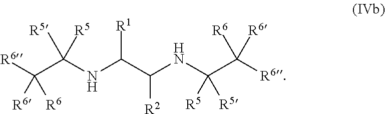

Embodiment 32 provides the method of Embodiment 29 or 30, wherein the cyclizing step comprises treatment of the compound of formula (IV) with HCl and either (CH₂O)ₙ at a temperature of about 60° C. or HC(OEt)₃ and HCO₂H at a temperature of about 125° C.

Embodiment 33 provides a method of promoting hydration of an alkyne, the method comprising contacting the alkyne and water in the presence of the compound of any one of Embodiments 13-28.

Embodiment 34 provides the method of Embodiment 33, wherein the alkyne is selected from the group consisting of optionally substituted C₂-C₁₂ alkynyl, optionally substituted C₆-C₁₀ aralkynyl, and optionally substituted C₄-C₁₀ heteroaralkynyl, wherein each optional substituent is at least one selected from the group consisting of C₁-C₆ alkoxy, C₁-C₆ haloalkoxy, C₁-C₆ haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, halogen, OH, NH₂, NH(C₁-C₆ alkyl), N(C₁-C₆ alkyl)₂, CN, NO₂, CHO, C(═O)OH, C(═O)O(C₁-C₆ alkyl), C(═O)NH₂, C(═O)NH(C₁-C₆ alkyl), and C(═O)N(C₁-C₆ alkyl)₂.

Embodiment 35 provides the method of Embodiment 33 or 34, wherein the contacting occurs in the presence of a Lewis acid.

Embodiment 36 provides the method of Embodiment 35, wherein the Lewis acid is selected from the group consisting of AgNTf₂, AgOAc, AgOTf, NaBArF, KB(C₆F₅)₄, and AgSbF₆.

Embodiment 37 provides the method of any one of Embodiments 33-36, wherein the contacting occurs in the presence of a solvent.

Embodiment 38 provides the method of Embodiment 37, wherein the solvent is 1,4-dioxane.

Embodiment 39 provides the method of any one of Embodiments 33-38, wherein the contacting occurs at a temperature of about 120° C.

Embodiment 40 provides the method of any one of Embodiments 33-39, wherein the compound of any one of Embodiments 13-28 is present in an amount ranging from about 0.1 to about 1.0 mol %.

Embodiment 41 provides a method of promoting a reaction between an alkyne and a borylation reagent, the method comprising contacting the alkyne and the borylation reagent in the presence of a base, a protic solvent or electrophile, and the compound of any one of Embodiments 13-28.

Embodiment 42 provides the method of Embodiment 41, wherein the alkyne is selected from the group consisting of optionally substituted C₂-C₁₂ alkynyl, optionally substituted C₆-C₁₀ aralkynyl, and optionally substituted C₄-C₁₀ heteroaralkynyl, wherein each optional substituent is at least one selected from the group consisting of C₁-C₆ alkoxy, C₁-C₆ haloalkoxy, C₁-C_{6,} haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, halogen, OH, NH₂, NH(C₁-C₆ alkyl), N(C₁-C₆ alkyl)₂, CN, NO₂, CHO, C(═O)OH, C(═O)O(C₁-C₆ alkyl), C(═O)NH₂, C(═O)NH(C₁-C₆ alkyl), and C(═O)N(C₁-C₆ alkyl)₂.

Embodiment 43 provides the method of Embodiment 41 or 42, wherein the borylation reagent is a diboronic ester.

Embodiment 44 provides the method of Embodiment 43, wherein the diboronic ester is B₂(pin)₂.

Embodiment 45 provides the method of any one of Embodiments 41-44, wherein the compound of any one of Embodiments 13-28 is present in an amount ranging from about 0.1 to about 2.0 mol %.

Embodiment 46 provides the method of any one of Embodiments 41-45, wherein the protic solvent is methanol (MeOH).

Embodiment 47 provides the method of any one of Embodiments 41-46, wherein the base is KOt-Bu.

Embodiment 48 provides the method of any one of Embodiments 41-47, wherein the electrophile is selected from the group consisting of C₁-C₁₂ haloalkyl, C₆-C₁₂ haloaralkyl, C₆-C₁₀ aryl halide, C₄-C₁₀ heteroaryl halide, C₁-C₁₂ alkyl-(C═O)Cl, C₆-C₁₀ aryl-(C═O)Cl, C₄-C₁₀ heteroaryl-(C═O)Cl, and CO₂.

Embodiment 49 provides the method of Embodiment 48, wherein the electrophile is methyl iodide (MeI).

Embodiment 50 provides the method of any one of Embodiments 41-49, wherein the contacting occurs at a temperature ranging from about 20 to about 70° C.

Embodiment 51 provides a method of promoting a reaction between a first reagent and a second reagent, the method comprising contacting the first reagent and the second reagent in the presence of a base and the compound of any one of Embodiments 13-28.

Embodiment 52 provides the method of Embodiment 51, wherein the first reagent is selected from the group consisting of optionally substituted C₆-C₁₀ aryl iodide and optionally substituted C₄-C₁₀ heteroaryl iodide, wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, N(C₁-C₆ alkyl)₂, CN, NO₂, C(═O)NH₂, C(═O)NH(C₁-C₆ alkyl), and C(═O)N(C₁-C₆ alkyl)₂.

Embodiment 53 provides the method of Embodiment 51 or 52, wherein the second reagent is a optionally substituted C₆-C₁₀ hydroxyaryl, wherein each optional substituent is at least one selected from the group consisting of C₁-C₆ alkoxy, C₁-C₆ haloalkoxy, C₁-C_{6,} haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, N(C₁-C₆ alkyl)₂, CN, NO₂, C(═O)NH₂, C(═O)NH(C₁-C₆ alkyl), and C(═O)N(C₁-C₆ alkyl)₂.

Embodiment 54 provides the method of any one of Embodiments 51-53, wherein the base is Cs₂CO₃.

Embodiment 55 provides the method of any one of Embodiments 51-54, wherein the contacting occurs in the presence of a solvent.

Embodiment 56 provides the method of Embodiment 55, wherein the solvent is dimethylformamide (DMF).

Embodiment 57 provides the method of any one of Embodiments 51-56, wherein the compound of any one of Embodiments 13-28 is present in an amount ranging from about 0.1 to about 2 mol %.

Embodiment 58 provides the method of any one of Embodiments 51-57, wherein the contacting occurs at a temperature ranging from about 140 to about 160° C.

Embodiment 59 provides a method of promoting a reaction between an aryl bromide and a second reagent, the method comprising contacting the aryl bromide and the second reagent in the presence of a base and the compound of any one of Embodiments 13-28.

Embodiment 60 provides the method of Embodiment 59, wherein the aryl bromide is selected from the group consisting of optionally substituted C₆-C₁₀ aryl bromide and optionally substituted C₄-C₁₀ heteroaryl bromide, wherein each optional substituent is at least one selected the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, N(C₁-C₆ alkyl)₂, CN, NO₂, C(═O)NH₂, C(═O)NH(C₁-C₆ alkyl), and C(═O)N(C₁-C₆ alkyl)₂.

Embodiment 61 provides the method of Embodiment 59 or 60, wherein the compound of any one of Embodiments 13-28 is present in an amount ranging from about 1 to about 5 mol %.

Embodiment 62 provides the method of any one of Embodiments 59-61, wherein the contacting occurs at a temperature ranging from about 70 to about 130° C.

Embodiment 63 provides the method of any one of Embodiments 59-62, wherein the base is selected from the group consisting of K₂CO₃ and KOt-Bu.

Embodiment 64 provides the method of any one of Embodiments 59-63, wherein the second reagent is a secondary amine.

Embodiment 65 provides the method of Embodiment 64, wherein the secondary amine is selected from the group consisting of optionally substituted C₄-C₁₂ heterocycloalkyl and optionally substituted NH(C₁-C₆ alkyl)₂, wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, N(C₁-C₆ alkyl)₂, CN, NO₂, C(═O)NH₂, C(═O)NH(C₁-C₆ alkyl), and C(═O)N(C₁-C₆ alkyl)₂.

Embodiment 66 provides the method of any one of Embodiments 59-65, wherein the contacting occurs in the presence of a protic solvent.

Embodiment 67 provides the method of Embodiment 66, wherein the protic solvent is isopropanol (i-PrOH).

Embodiment 68 provides the method of Embodiment 66 or 67, wherein the second reagent is selected from the group consisting of optionally substituted C₆-C₁₀ aryl boronic acid and optionally substituted C₄-C₁₀ heteroaryl boronic acid, wherein each optional substituent is at least one selected the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, N(C₁-C₆ alkyl)₂, CN, NO₂, C(═O)NH₂, C(═O)NH(C₁-C₆ alkyl), and C(═O)N(C₁-C₆ alkyl)₂.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A compound of formula (I): 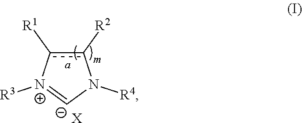
wherein:
X is a counter anion;
R¹ and R² are each independently selected from the group consisting of H, optionally substituted C₁-C₆ alkyl, optionally substituted C₂-C₆ alkenyl, optionally substituted benzyl, optionally substituted phenyl, and optionally substituted naphthyl, and optionally substituted C₄-C₁₀ heteroaryl, or
R¹ and R² may combine with the ring to which they are attached to form C₅-C₁₂ cycloalkyl, C₆-C₁₀ aryl, or C₄-C₁₀ heteroaryl,
wherein each optional substituent in R¹ and R² is independently at least one substituent selected from the group consisting of C₁-C₃ alkyl, C₂-C₆ alkenyl, phenyl, naphthyl, C₄-C₁₀ heteroaryl, N(R^{a})(R^{b}), OR^{b}, CN, CF₃, OCF₃, C(═O)R^{b}, N(R)S(═O)₂R^{b}, C(═O)N(R^{a})(R^{b}), and C(═O)OR^{b};
R³ and R⁴ are each independently 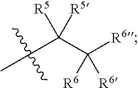
R⁵ and R^{5′} are each independently selected from the group consisting of C₁-C₆ alkyl, C₃-C₁₂ cycloalkyl, C₂-C₁₀ heterocycloalkyl, C₆-C₁₀ aryl, C₄-C₁₀ heteroaryl, halogen, OR^{b}, N(R^{a})(R^{b}), SR^{b}, and Si(R^{b})₃,
wherein R⁵ and R^{5′} may combine with the carbon atom to which they are bound to form one selected from the group consisting of C₃-C₈ cycloalkyl and C₂-C₇ heterocycloalkyl;
R⁶, R^{6′}, and R^{6″} are each independently selected from the group consisting of H, C₁-C₆ alkyl, C₃-C₁₂ cycloalkyl, C₂-C₁₀ heterocycloalkyl, C₆-C₁₀ aryl, C₄-C₁₀ heteroaryl, halogen, OR^{b}, N(R^{a})(R^{b}), SR^{b}, and Si(R^{b})₃,
wherein at least two of R⁶, R^{6′}, and R^{6″} may combine with the carbon atom to which they are bound to form one selected from the group consisting of C₃-C₁₂ cycloalkyl, C₂-C₁₀ heterocycloalkyl, C₅-C₁₂ fused cycloalkyl, C₄-C₁₀ fused heterocycloalkyl, C₅-C₁₂ bridged cycloalkyl, and C₄-C₁₀ bridged heterocycloalkyl,
wherein any one of R⁶, R^{6′}, and R^{6″} may combine with any one of R⁵ and R^{5′} and the carbon atoms to which they are bound to form one selected from the group consisting of C₄-C₁₂ cycloalkyl and C₃-C₁₀ heterocycloalkyl, and
wherein if R⁵ and R^{5′} are Me, and two of R⁶, R^{6′}, and R^{6″} are H, then the other one of R⁶, R^{6′}, and R^{6″} is not H or Me;
wherein if R⁵ and R^{5′} are Me, two of R⁶, R^{6′}, and R^{6″} are H, and one of R⁶, R^{6′}, and R^{6″} is t-Bu, then M is not BF₄;
bond a is a single or double bond;
each occurrence of R^{a} is independently H or C₁-C₃ alkyl;
each occurrence of R^{b} is independently selected from the group consisting of H, C₁-C₆ alkyl, C₁-C₃ haloalkyl, C₂-C₆ alkenyl, benzyl, naphthyl, C₄-C₁₀ heteroaryl, and phenyl optionally substituted with at least one substituent selected from the group consisting of C₁-C₃ alkyl and halogen; and
m is 1 or 2.

2. The compound of claim 1, wherein at least one of the following applies:
(a) X is selected from the group consisting of H, halide, OS(═O)₂R^{b}, OC(═O)R^{b}, N(C(═O)R^{b})₂, tetracoordinate boronate, and hexacoordinate phosphorus;
(b) at least one of R⁵ and R^{5′} is Me or Et;
(c) at least two of R⁶, R^{6′}, and R^{6″} are H;
(d) at least one of R⁶, R^{6′}, and R^{6″} is tert-butyl or Me;
(e) at least one of R¹ and R² is H or Me;
(f) at least one of R³ and R⁴ is 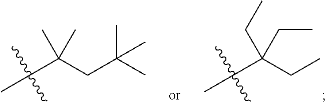
and
(g) m is 1.

3. The compound of claim 2, wherein at least one of the following applies:
(a) X is Cl or BF₄;
(b) R¹ and R² are identical; and
(c) R³ and R⁴ are identical.

4-11. (canceled)

12. The compound of claim 1, which is selected from the group consisting of:
1,3-bis(2,4,4-trimethylpentan-2-yl)-1H-imidazol-3-ium chloride;
1,3-bis(2,4,4-trimethylpentan-2-yl)-1H-imidazol-3-ium tetrafluoroborate;
1,3-bis(2,4,4-trimethylpentan-2-yl)-4,5-dihydro-1H-imidazol-3-ium chloride;
1,3-bis(2,4,4-trimethylpentan-2-yl)-1H-benzo[d]imidazol-3-ium chloride;
1,3-bis(3-ethylpentan-3-yl)-1H-imidazol-3-ium chloride; and
1,3-bis(3-ethylpentan-3-yl)-4,5-dihydro-1H-imidazolium chloride.

13. A compound of formula (II): 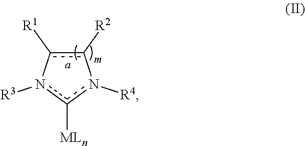
wherein:
M is a transition metal;
L is a ligand of M, wherein each occurrence of L can be the same or different;
R¹ and R² are each independently selected from the group consisting of H, optionally substituted C₁-C₆ alkyl, optionally substituted C₂-C₆ alkenyl, optionally substituted benzyl, optionally substituted phenyl, and optionally substituted naphthyl, and optionally substituted C₄-C₁₀ heteroaryl, or
R¹ and R² may combine with the ring to which they are attached to form C₅-C₁₂ cycloalkyl, C₆-C₁₀ aryl, or C₄-C₁₀ heteroaryl,
wherein each optional substituent in R¹ and R² is independently at least one substituent selected from the group consisting of C₁-C₃ alkyl, C₂-C₆ alkenyl, phenyl, naphthyl, C₄-C₁₀ heteroaryl, N(R^{a})(R^{b}), OR^{b}, CN, CF₃, OCF₃, C(═O)R^{b}, N(R)S(═O)₂R^{b}, C(═O)N(R^{a})(R^{b}), and C(═O)OR^{b};
R³ and R⁴ are each independently 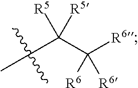
R⁵ and R^{5′} are each independently selected from the group consisting of C₁-C₆ alkyl, C₃-C₁₂ cycloalkyl, C₂-C₁₀ heterocycloalkyl, C₆-C₁₀ aryl, C₄-C₁₀ heteroaryl, halogen, OR^{b}, N(R^{a})(R^{b}), SR^{b}, and Si(R^{b})₃,
wherein R⁵ and R^{5′} may combine with the carbon atom to which they are bound to form one selected from the group consisting of C₃-C₈ cycloalkyl and C₂-C₇ heterocycloalkyl;
R⁶, R^{6′}, and R^{6″} are each independently selected from the group consisting of H, C₁-C₆ alkyl, C₃-C₁₂ cycloalkyl, C₂-C₁₀ heterocycloalkyl, C₆-C₁₀ aryl, C₄-C₁₀ heteroaryl, halogen, OR^{b}, N(R^{a})(R^{b}), SR^{b}, and Si(R^{b})₃,
wherein at least two of R⁶, R^{6′}, and R^{6″} may combine with carbon atom to which they are bound to form one selected from the group consisting of C₃-C₁₂ cycloalkyl, C₂-C₁₀ heterocycloalkyl, C₅-C₁₂ fused cycloalkyl, C₄-C₁₀ fused heterocycloalkyl, C₅-C₁₂ bridged cycloalkyl, and C₄-C₁₀ bridged heterocycloalkyl,
wherein any one of R⁶, R^{6′}, and R^{6″} may combine with any one of R⁵ and R^{5′} and the carbon atoms to which they are bound to form one selected from the group consisting of C₄-C₁₂ cycloalkyl and C₃-C₁₀ heterocycloalkyl, and
wherein if R⁵ and R^{5′} are Me, and two of R⁶, R^{6′}, and R^{6″} are H, then the other one of R⁶, R^{6′}, and R^{6″} is not H or Me;
each occurrence of [symbol] is a single or double bond,
wherein no more than one [symbol] bonding a C atom and a N atom is a double bond;
each occurrence of R^{a} is independently H or C₁-C₃ alkyl;
each occurrence of R^{b} is independently selected from the group consisting of H, C₁-C₆ alkyl, C₁-C₃ haloalkyl, C₂-C₆ alkenyl, benzyl, naphthyl, C₄-C₁₀ heteroaryl, and phenyl optionally substituted with at least one substituent selected from the group consisting of C₁-C₃ alkyl and halogen;
n is an integer which is selected from the group consisting of 1, 2, 3, 4, and 5; and
m is an integer which is either 1 or 2.

14. The compound of claim 13, wherein at least one of the following applies:
(a) M is selected from the group consisting of Cu, Ag, Au, Pd, Ni, Pt, Co, Rh, Ir, Fe, Ru, and Os;
(b) at least one of R⁵ and R^{5′} is Me or Et;
(c) at least two of R⁶, R^{6′}, and R^{6″} are H;
(d) at least one of R⁶, R^{6′}, and R^{6″} is tert-butyl or Me;
(e) at least one of R¹ and R² is H or Me;
(f) at least one of R³ and R⁴ is 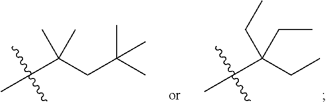
(g) m is 1;
(h) R¹ and R² are identical; and
R³ and R⁴ are identical.

15. The compound of claim 13, wherein one of the following applies:
(a) L is an anionic ligand, optionally wherein the anionic ligand is selected from the group consisting of H, OS(═O)₂R^{b}, OC(═O)R^{b}, N(C(═O)R^{b})₂, halogen, C₆-C₁₀ aryl, and C₄-C₁₀ heteroaryl; or
(b) L is a neutral ligand, optionally wherein the neutral ligand is pyridyl optionally substituted with at least one halogen or a C₂-C₁₂ alkene.

16. (canceled)

17. The compound of claim 15, wherein one of the following applies:
(a) the anionic ligand is Cl; or
(b) the neutral ligand is

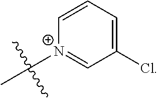 [SMILES: *[n+]1cccc(Cl)c1]

18-27. (canceled)

28. The compound of claim 13, which is selected from the group consisting of:
1,3-bis(2,4,4-trimethylpentan-2-yl)-2,3-dihydro-1H-imidazol-2-ylidene gold(I) chloride;
1,3-bis(2,4,4-trimethylpentan-2-yl)-2,3-dihydro-1H-imidazol-2-ylidene copper(I) chloride;
1,3-bis(2,4,4-trimethylpentan-2-yl)-2,3-dihydro-1H-imidazol-2-ylidene silver(I) chloride;
1,3-bis(2,4,4-trimethylpentan-2-yl)-2,3-dihydro-1H-imidazol-2-ylidene palladium(II)(allyl) chloride;
3-chloropyridine [1,3-bis(2,4,4-trimethylpentan-2-yl)-2,3-dihydro-1H-imidazol-2-ylidene] palladium(II) dichloride;
1,3-bis(2,4,4-trimethylpentan-2-yl)imidazolidin-2-ylidene gold(I) chloride;
1,3-bis(2,4,4-trimethylpentan-2-yl)imidazolidin-2-ylidene palladium(II) (allyl) chloride;
1,3-bis(2,4,4-trimethylpentan-2-yl)benzimidazolidin-2-ylidene gold(I) chloride;
1,3-bis(2,4,4-trimethylpentan-Z-yObenzimidazolidin-2-ylidene copper(I) chloride;
3-chloropytidine [1,3-bis(2,4,4-trimethylpentan-2-yl)-benzimidazol-2-ylidene] palladium(II) dichloride
allyl [1,3-bis(2,4,4-trimethylpentan-2-yl)benzimidazolidin-2-ylidene] palladium(II) chloride;
1,3-bis(3-ethylpentan-3-yl)-1H-imidazol-2-ylidene gold(I) chloride;
1,3-bis(3-ethylpentan-3-yl)-1H-imidazol-2-ylidene copper(I) chloride;
1,3-bis(3-ethylpentan-3-yl)-1H-imidazol-2-ylidene silver(I) chloride;
3-chloropyridine [1,3-bis(3-ethylpentan-3-yl)-1H-imidazol-2-ylidene] palladium(II) dichloride;
1,3-bis(3-ethylpentan-3-yl)-4,5-dihydro-1H-imidazol-2-ylidene gold(I) chloride; and
1,3-bis(3-ethylpentan-3-yl)-4,5-dihydro-1H-imidazol-2-ylidene copper(I) chloride.

29. A method of making the compound of formula (I) of claim 1, the method comprising:
contacting a compound of formula (III): 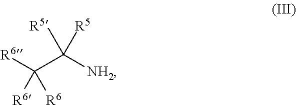
with (CHO)₂ to form a compound of formula (IV): 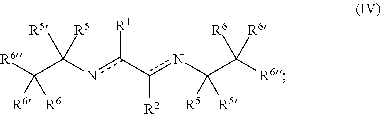
and
cyclizing the compound of formula (IV) to form the compound of formula (I) of claim 1.

30. The method of claim 29, wherein one of the following applies:
(a) R³ and R⁴ in the compound of formula (I) are identicali
(b) the compound of formula (IV) is treated with NaBH₄ to form a diamine compound of formula (IVb): 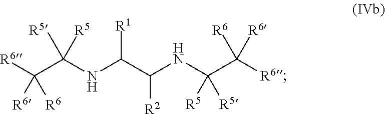
and
(c) the cyclizing step comprises treatment of the compound of formula (IV) with HCl and either (CH₂O)ₙ at a temperature of about 60° C. or HC(OEt)₃ and HCO₂H at a temperature of about 125° C.

31-32. (canceled)

33. A method of promoting hydration of an alkyne, the method comprising contacting the alkyne and water in the presence of the compound of claim 13.

34. The method of claim 33, wherein at least one of the following applies:
(a) the alkyne is selected from the group consisting of optionally substituted C₂-C₁₂ alkynyl, optionally substituted C₆-C₁₀ aralkynyl, and optionally substituted C₄-C₁₀ heteroaralkynyl, wherein each optional substituent is at least one selected from the group consisting of C₁-C₆ alkoxy, C₁-C₆ haloalkoxy, C₁-C₆ haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, halogen, OH, NH₂, NH(C₁-C₆ alkyl), N(C₁-C₆ alkyl)₂, CN, NO₂, CHO, C(═O)OH, C(═O)O(C₁-C₆ alkyl), C(═O)NH₂, C(═O)NH(C₁-C₆ alkyl), and C(═O)N(C₁-C₆ alkyl)₂;
(b) the contacting occurs in the presence of a Lewis acid, optionally wherein the Lewis acid is selected from the group consisting of AgNTf₂, AgOAc, AgOTf, NaBArF, KB(C₆F₅)₄, and AgSbF₆;
(c) the contacting occurs in the presence of a solvent, optionally wherein the solvent is 1,4-dioxane;
(d) the contacting occurs at a temperature of about 120° C.; and
(e) the compound is present in an amount ranging from about 0.1 to about 1.0 mol %.

35-40. (canceled)

41. A method of promoting a reaction between an alkyne and a borylation reagent, the method comprising contacting the alkyne and the borylation reagent in the presence of a base, a protic solvent or electrophile, and the compound of claim 13.

42. The method of claim 41, wherein at least one of the following applies:
(a) the alkyne is selected from the group consisting of optionally substituted C₂-C₁₂ alkynyl, optionally substituted C₆-C₁₀ aralkynyl, and optionally substituted C₄-C₁₀ heteroaralkynyl, wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, halogen, OH, NH₂, NH(C₁-C₆ alkyl), N(C₁-C₆ alkyl)₂, CN, NO₂, CHO, C(═O)OH, C(═O)O(C₁-C₆ alkyl), C(═O)NH₂, C(═O)NH(C₁-C₆ alkyl), and C(═O)N(C₁-C₆ alkyl)₂;
(b) the borylation reagent is a diboronic ester, optionally wherein the diboronic ester is B₂(pin)₂;
(c) the compound is present in an amount ranging from about 0.1 to about 2.0 mol %;
(d) the protic solvent is methanol (MeOH);
(e) the base is KOt-Bu;
(f) the electrophile is selected from the group consisting of C₁-C₁₂ haloalkyl, C₆-C₁₂ haloaralkyl, C₆-C₁₀ aryl halide, C₄-C₁₀ heteroaryl halide, C₁-C₁₂ alkyl-(C═O)Cl, C₆-C₁₀ aryl-(C═O)Cl, C₄-C₁₀ heteroaryl-(C=0)Cl, and CO₂, optionally wherein the electrophile is methyl iodide (MeI); and
(g) the contacting occurs at a temperature ranging from about 20 to about 70° C.

43-50. (canceled)

51. A method of promoting a reaction between a first reagent and a second reagent, the method comprising contacting the first reagent and the second reagent in the presence of a base and the compound of claim 13.

52. The method of claim 51, wherein at least one of the following applies:
(a) the first reagent is selected from the group consisting of optionally substituted C₆-C₁₀ aryl iodide and optionally substituted C₄-C₁₀ heteroaryl iodide, wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, N(C₁-C₆ alkyl)₂, CN, NO₂, C(═O)NH₂, C(═O)NH(C₁-C₆ alkyl), and C(═O)N(C₁-C₆ alkyl)₂;
(b) the second reagent is a optionally substituted C₆-C₁₀ hydroxyaryl, wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, N(C₁-C₆ alkyl)₂, CN, NO₂ C(═O)NH₂, C(═O)NH(C₁-C₆ alkyl), and C(═O)N(C₁-C₆ alkyl)₂;
(c) the base is Cs₂CO₃;
(d) the contacting occurs in the presence of a solvent, optionally wherein the solvent is dimethylformamide (DMF);
(e) the contacting occurs at a temperature ranging from about 0.1 to about 2 mol %; and
(f) the contacting occurs at a temperature ranging from about 140 to about 160° C.

53-58. (canceled)

59. A method of promoting a reaction between an aryl bromide and a second reagent, the method comprising contacting the aryl bromide and the second reagent in the presence of a base and the compound of claim 13.

60. The method of claim 59, wherein at least one of the following applies:
(a) the aryl bromide is selected from the group consisting of optionally substituted C₆-C₁₀ aryl bromide and optionally substituted C₄-C₁₀ heteroaryl bromide, wherein each optional substituent is at least one selected the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, N(C₁-C₆ alkyl)₂, CN, NO₂, C(═O)NH₂, C(═O)NH(C₁-C₆ alkyl), and C(═O)N(C₁-C₆ alkyl)₂;
(b) the compound is present in an amount ranging from about 1 to about 5 mol %;
(c) the contacting occurs at a temperature ranging from about 70 to about 130° C.;
(d) the base is selected from the group consisting of K₂CO₃ and KOt-Bu;
(e) the second reagent is a secondary amine, optionally wherein the secondary amine is selected from the group consisting of optionally substituted C₄-C₁₂ heterocycloalkyl and optionally substituted NH(C₁-C₆ alkyl), wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, N(C₁-C₆ alkyl)₂, CN, NO₂, C(═O)NH₂, C(═O)NH(C₁-C₆ alkyl), and C(═O)N(C₁-C₆ alkyl)₂; and
(f) the contacting occurs in the presence of a protic solvent, optionally wherein the protic solvent is isopropanol (i-PrOH).

61-65. (canceled)

66. The method of claim 59, wherein the contacting occurs in the presence of a protic solvent, optionally wherein one of the following applies:
(a) the compound is present in an amount ranging from about 1 to about 5 mol %;
(b) the protic solvent is isopropanol (i-PrOH);
(c) the contacting occurs at a temperature ranging from about 70 to about 130° C.;
(d) the base is selected from the group consisting of K₂CO₃ and KOt-Bu; and
(e) the second reagent is selected from the group consisting of optionally substituted C₆-C₁₀ aryl boronic acid and optionally substituted C₄-C₁₀ heteroaryl boronic acid, wherein each optional substituent is at least one selected the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, N(C₁-C₆ alkyl)₂, CN, NO₂ C(═O)NH₂, C(═O)NH(C₁-C₆ alkyl), and C(═O)N(C₁-C₆ alkyl)₂.

67-68. (canceled)
